(19) United States
(12) Patent Application Publication
(10) Pub. No.: US 2026/0026806 A1
(43) Pub. Date: Jan. 29, 2026

(54) SYSTEM AND METHOD FOR REGULATING ARTICULATION LOADS IN SURGICAL STAPLER

(71) Applicant: Cilag GmbH International, Zug (CH)

(72) Inventors: Shiv Wadwani, San Jose, CA (US); Maximilian E. Reese, Los Gatos, CA (US); Paul Moubarak, West Chester, OH (US)

(21) Appl. No.: 18/780,913

(22) Filed: Jul. 23, 2024

(51) Int. Cl. A61B 17/072; A61B 17/00; A61B 90/00; G16H 40/63

(52) U.S. Cl. CPC A61B 17/07207; A61B 90/06; G16H 40/63; A61B 2017/00022; A61B 2017/00398; A61B 2017/00477; A61B 2017/07257; A61B 2017/07271; A61B 2017/07278; A61B 2017/07285; A61B 2090/064

(57) ABSTRACT

A method for performing an electrosurgical procedure using an electrosurgical instrument. The method includes selecting a desired pose of an end effector with respect to a shaft assembly and conducting a surgical procedure on a patient tissue via the end effector that causes tensions to be exerted on a plurality of articulation cables for the desired pose of the end effector. The method further includes obtaining, by a controller via a plurality of sensors, the tensions that are exerted on the plurality of articulation cables during the surgical procedure and determining whether the tensions exceed a first predetermined tension threshold and in response to determining that the tensions exceed the first predetermined tension threshold, operating at least one motor of the plurality of motors to reduce the tensions until the tensions are at or below the first predetermined tension threshold.

### BACKGROUND

In some settings, endoscopic surgical instruments may be preferred over traditional open surgical devices to minimize the size of the surgical incision as well as post-operative recovery time and complications. Consequently, some endoscopic surgical instruments may be suitable for placement of a distal end effector at a desired surgical site through the cannula of a trocar. These distal end effectors may engage tissue in a number of ways to achieve a diagnostic or therapeutic effect (e.g., endocutter, grasper, cutter, stapler, clip applier, access device, drug/gene therapy delivery device, and energy delivery device using ultrasound, RF, laser, etc.). Endoscopic surgical instruments may include a shaft that extends proximally from the end effector to a handle portion that is manipulated by the clinician, or alternatively to a robot. Such a shaft may enable insertion to a desired depth and rotation about the longitudinal axis of the shaft, thereby facilitating positioning of the end effector within the patient. Positioning of an end effector may be further facilitated through inclusion of one or more articulation joints or features, enabling the end effector to be selectively articulated or otherwise deflected relative to the longitudinal axis of the shaft.

Examples of endoscopic surgical instruments include surgical staplers. Some such staplers are operable to clamp down on layers of tissue, cut through the clamped layers of tissue, and drive staples through the layers of tissue to substantially seal the severed layers of tissue together near the severed ends of the tissue layers. Such endoscopic surgical staplers may also be used in open procedures and/or other non-endoscopic procedures. By way of example only, a surgical stapler may be inserted through a thoracotomy and thereby between a patient's ribs to reach one or more organs in a thoracic surgical procedure that does not use a trocar as a conduit for the stapler. Such procedures may include the use of the stapler to sever and close a vessel leading to an organ, such as a lung. For instance, the vessels leading to an organ may be severed and closed by a stapler before removal of the organ from the thoracic cavity. Of course, surgical staplers may be used in various other settings and procedures.

It is desirable to reduce the force spikes encountered by a surgical instrument drivetrain during a surgical procedure. While various kinds of surgical staplers and associated components have been made and used, it is believed that no one prior to the inventor(s) has made or used the invention described in the appended claims.

### BRIEF DESCRIPTION OF THE DRAWINGS

The accompanying drawings, which are incorporated in and constitute a part of this specification, illustrate examples of the invention, and, together with the general description of the invention given above, and the detailed description of the examples given below, serve to explain the principles of the present invention.

FIG. 1 is a perspective view of an illustrative surgical instrument having a housing, a shaft assembly, an articulation joint, an end effector, a motor, sensors, and a controller;

FIG. 2 is a partial perspective view of the surgical instrument of FIG. 1, with select components omitted from view to reveal portions of a cable articulation subsystem, a knife firing subsystem, and a roll subsystem of the surgical instrument;

FIG. 3 is an enlarged perspective view of the end effector and the articulation joint of the surgical instrument of FIG. 1;

FIG. 4 is an exploded view of a distal end portion of the surgical instrument of FIG. 1;

FIG. 5 is an enlarged perspective view of a knife of the end effector of the surgical instrument of FIG. 1;

FIG. 6 is an end view of the end effector of FIG. 3;

FIG. 7 is an enlarged perspective view of the end effector and the articulation joint of FIG. 3, with an anvil of the end effector omitted;

FIG. 8A is a side cross-sectional view of a distal end portion of the surgical instrument of FIG. 1, depicting the anvil in an open position;

FIG. 8B is a side cross-sectional view of the distal end portion of the surgical instrument of FIG. 1, depicting the anvil in a grasping position with the knife partially advanced;

FIG. 8C is a side cross-sectional view of the distal end portion of the surgical instrument of FIG. 1, depicting the anvil in a clamping position with the knife partially advanced;

FIG. 8D is a side cross-sectional view of the distal end portion of the surgical instrument of FIG. 1, depicting the anvil in the clamping position with the knife fully advanced;

FIG. 9A is an enlarged side cross-sectional view of a proximal end portion of the end effector of the surgical instrument of FIG. 1, depicting the anvil in the open position;

FIG. 9B is an enlarged side cross-sectional view of the proximal end portion of the end effector of the surgical instrument of FIG. 1, depicting the anvil in a grasping position with the knife partially advanced;

FIG. 9C is an enlarged side cross-sectional view of the proximal end portion of the end effector of the surgical instrument of FIG. 1, depicting the anvil in a clamping position with the knife partially advanced;

FIG. 9D is an enlarged side cross-sectional view of the proximal end portion of the end effector of the surgical instrument of FIG. 1, depicting the anvil in the clamping position with the knife fully advanced;

FIG. 10 is an exploded perspective view of the articulation joint of the surgical instrument of FIG. 1;

FIG. 11 is an end view of the articulation joint of FIG. 10;

FIG. 12 is a cross-sectional view of a portion of the articulation joint of FIG. 10, taken along line 12-12 in FIG. 11;

FIG. 13 is a cross-sectional view of a portion of the articulation joint of FIG. 10, taken along line 13-13 in FIG. 11;

FIG. 14 is a perspective view of the distal end of the surgical instrument of FIG. 1, depicting the end effector articulated vertically and laterally with the anvil open;

FIG. 15 is a side view of the distal end of the surgical instrument of FIG. 1, depicting the end effector articulated vertically with the anvil closed;

FIG. 16 is a top view of the distal end of the surgical instrument of FIG. 1, depicting the end effector articulated laterally with the anvil closed;

FIG. 17 is an exploded perspective view of a portion of the surgical instrument of FIG. 1, depicting portions of the cable articulation subsystem, the knife firing subsystem, and the roll subsystem;

FIG. 18 is a top view of a proximal end of the surgical instrument of FIG. 1, depicting portions of the cable articulation subsystem, the knife firing subsystem, and the roll subsystem;

FIG. 19 is a perspective view of a shaft assembly, a differential, and a firing rod of the surgical instrument of FIG. 1;

FIG. 20 depicts a flow chart of an illustrative method for controlling the surgical instrument of FIG. 1, according to one example;

FIG. 21 depicts a block diagram of an architecture of a feedback control system that embodies the illustrative method of FIG. 20;

FIG. 22 depicts a flow chart of an illustrative method for controlling the surgical instrument of FIG. 1, according to another example; and

FIG. 23 depicts a flow chart of an illustrative method for controlling the surgical instrument of FIG. 1, according to yet another example.

### DETAILED DESCRIPTION

The following detailed description should be read with reference to the drawings, in which like elements in different drawings are identically numbered. The drawings, which are not necessarily to scale, depict selected versions and are not intended to limit the scope of the invention. The detailed description illustrates by way of example, not by way of limitation, the principles of the invention. This description will clearly enable one skilled in the art to make and use the invention, and describes several versions, adaptations, variations, alternatives and uses of the invention, including what is presently believed to be the best mode of carrying out the invention.

Numerous specific details are set forth to provide a thorough understanding of the overall structure, function, manufacture, and use of the versions as described in the specification and illustrated in the accompanying drawings. Well-known operations, components, and elements have not been described in detail so as not to obscure the versions described in the specification. The reader will understand that the versions described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The terms “proximal” and “distal” are used herein with reference to a robotic platform manipulating the housing portion of the surgical instrument. The term “proximal” refers to the portion closest to the robotic platform and the term “distal” refers to the portion located away from the robotic platform. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Furthermore, the terms “about,” “approximately,” “substantially,” and the like as used herein in connection with any numerical values, ranges of values, and/or geometric/positional quantifications are intended to encompass the exact value(s) or quantification(s) referenced as well as a suitable tolerance that enables the referenced feature or combination of features to function for the intended purpose described herein. For example, “substantially parallel” encompasses nominally parallel structures, and “substantially equal” values encompass nominally equal values.

Furthermore, the use of “couple”, “coupled”, or similar phrases should not be construed as being limited to a certain number of components or a particular order of components unless the context clearly dictates otherwise.

### I. Overview of Illustrative Surgical Instrument

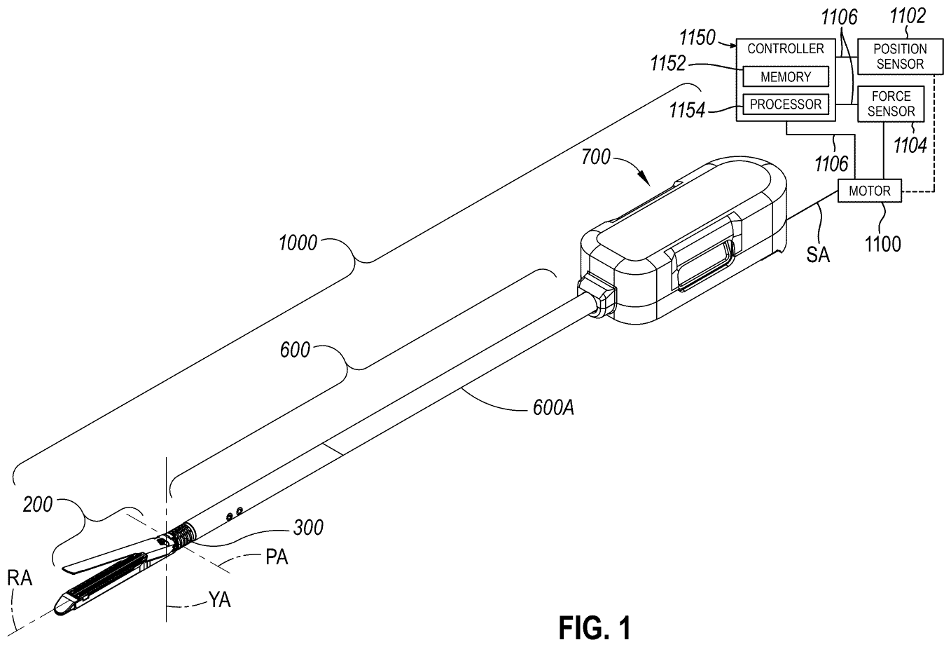

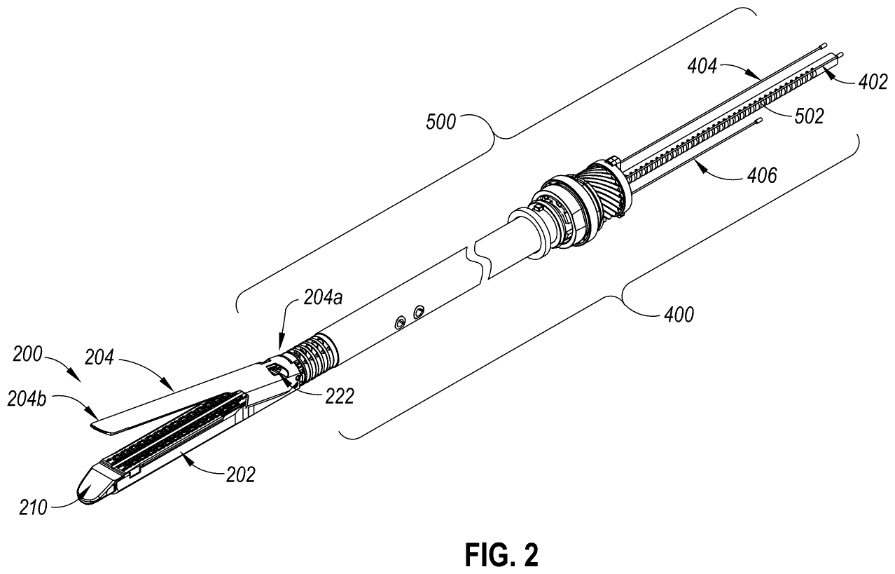

FIGS. 1-2 show an illustrative surgical instrument 1000 that is configured to grasp, clamp, incise, and seal patient tissue with staples. The surgical instrument 1000 comprises an end effector 200, an articulation joint 300 (also referred to as a “continuum joint”), an articulation drive subsystem 400 configured to articulate the end effector 200 via the articulation joint 300, a knife firing subsystem 500 configured to actuate the end effector 200 between various positions (e.g., an open position, a grasping position, and a clamping position) and to incise and staple patient tissue, a roll subsystem 600 configured to rotate the end effector 200 about a roll axis RA, and a housing 700.

As shown in FIG. 1, the surgical instrument 1000 additionally includes at least one motor (shown as motor 1100), a plurality of sensors (shown as including a position sensor 1102 and a force sensor 1104) and a controller 1150. The controller 1150 includes a memory 1152 and a processor 1154. The controller 1150 may include a counter, or alternatively, a separate counter may be incorporated separate from the controller 1150. As shown, the controller 1150 is in communication with the motor 1100, the position sensor 1102, and the force sensor 1104 using wires 1106. The force sensor 1104 provides power to the knife firing subsystem 500. The force sensor 1104 is configured to sense the force exerted by the motor 1100 on the knife firing subsystem 500. The position sensor 1102 may optionally be in communication with the motor 1100. In some versions, the force sensor 1104 comprises a torque sensor (such as a load cell) configured to sense torque of the motor 1100. The force sensor 1104 may be colinear with the shaft axis (SA).

With reference to FIGS. 1-2, the shaft assembly (e.g., a shaft) 600A extends proximally from the end effector 200 along a shaft axis SA. The housing 700 extends proximally from the shaft assembly 600A. The motor 1100, the position sensor 1102, and the force sensor 1104 are sized and configured to be positioned within the housing 700. The motor 1100 is coaxially positioned along the shaft axis SA. The motor 1100 is configured to actuate a knife 206 along a firing stroke using the knife firing subsystem 500 while the staple cartridge 210 is housed within the first jaw 202 to thereby cut tissue clamped by the first and second jaws 202, 204.

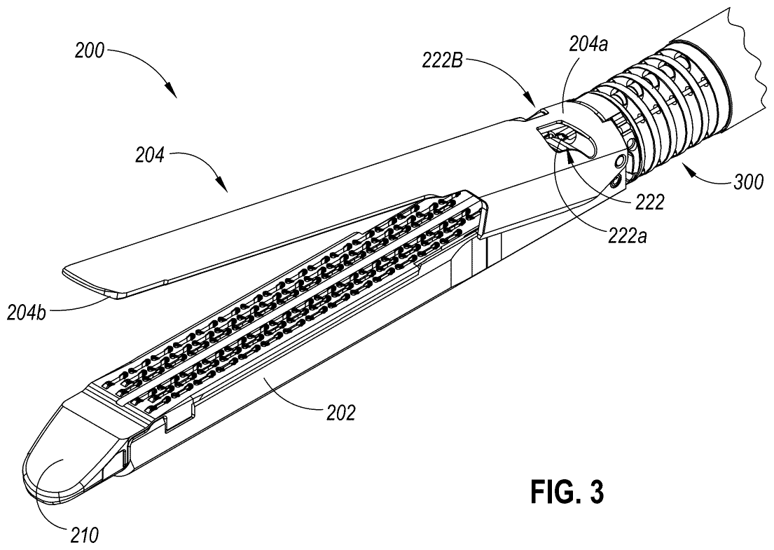

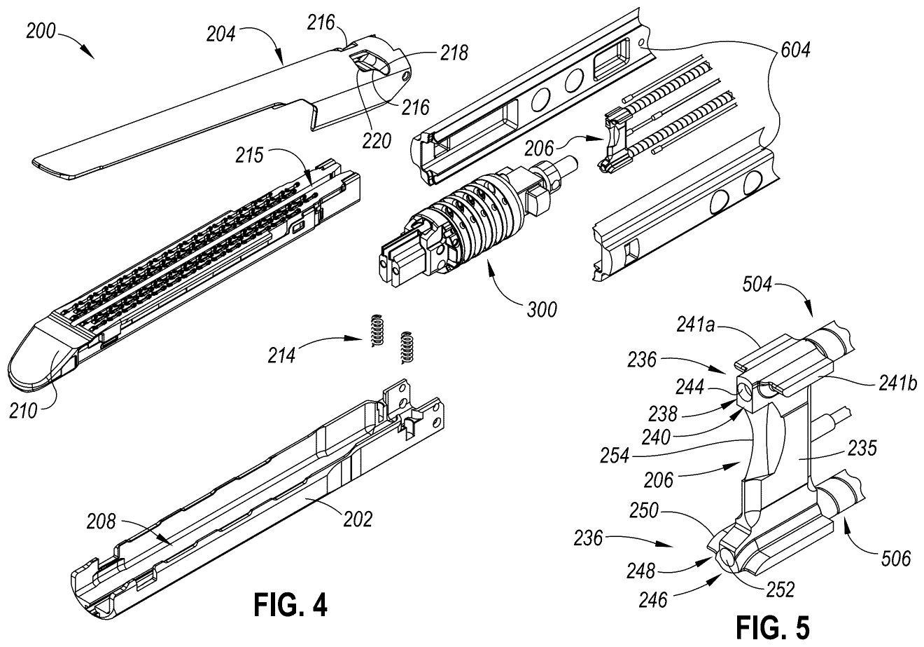

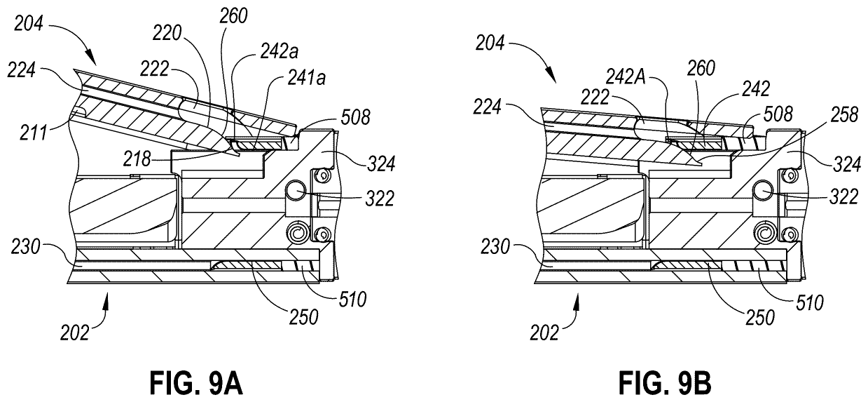

As shown best in FIGS. 3-4, the end effector 200 comprises a first jaw 202 (also known as a “cartridge jaw” or a “channel”) and a second jaw 204 (also known as an “anvil jaw” or just “anvil”) movable relative to the cartridge jaw 202 between an open position and a closed position. The cartridge jaw 202 and anvil jaw 204 may be elongated in form. The cartridge jaw 202 defines an elongated channel 208 for receiving a staple cartridge 210 (also known as a “reload”). The end effector 200 is operable to clamp, staple, and cut tissue. The end effector 200 includes the first jaw 202, the second jaw 204, and the knife 206. The first jaw 202 is configured to selectively receive a staple cartridge 210. The second jaw 204 includes a plurality of staple forming pockets 211 (see FIG. 9A).

At least one of the first or second jaws 202, 204 includes a ramp surface 216. As shown in FIGS. 4 and 9A-9D, the second jaw 204 includes the ramp surface 216. The ramp surface 216 is integrally formed together as a unitary piece together with the second jaw 204. Alternatively, the ramp surface 216 may be separately formed from the second jaw 204 and subsequently coupled with the second jaw 204. The ramp surface 216 includes a concave portion 258 and a convex portion 260. As shown, the concave portion 258 is proximal to the convex portion 260.

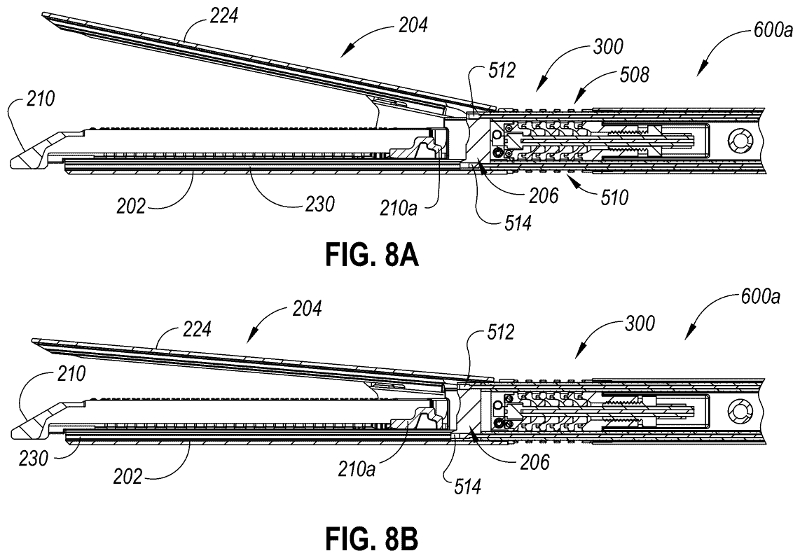

The anvil jaw 204 has a proximal end 204A, a distal end 204B, and a ramp surface 216 defined at the proximal end 204A, which is described in greater detail below with respect to FIGS. 4 and 9A-9D. The cartridge jaw 202 and anvil jaw 204 are pivotally coupled via a pivot pin 212 that extends through the cartridge jaw 202 and the anvil jaw 204. As seen in FIG. 7, one or more biasing springs 214 extend between the cartridge jaw 202 and anvil jaw 204 to bias the anvil jaw 204 to the open position.

The ramp surface 216 may be visible via an opening 222 (which may be formed as part of the manufacturing process to make the ramp surface 216) that has a first lateral end 222A and a second lateral end 222B and can be generally reniform shaped. In other words, the opening 222 may be open at its lateral ends 222A, 222B (FIG. 3). As seen in FIG. 4, the ramp surface 216 forms a lower surface of the opening 222. The ramp surface 216 can be arcuately shaped. For example, as shown particularly in FIGS. 4 and 9A-9D, it may be upwardly sloped at a first angle 218 and arcuately taper, in a distal direction, to a substantially horizontal second angled surface 220.

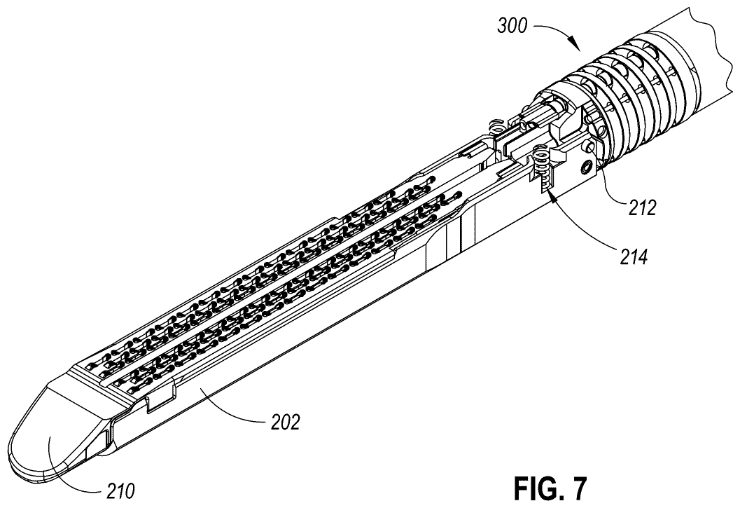

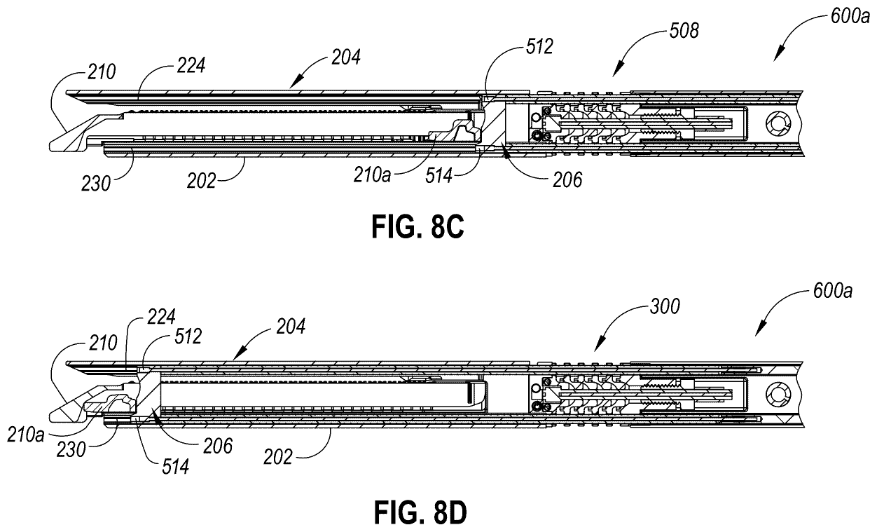

The anvil jaw 204 further defines a longitudinally extending upper knife channel 224 (see e.g., FIG. 8A, etc.). As shown particularly in FIG. 6, the upper knife channel 224 includes a centrally disposed cylindrical upper knife channel portion 226 and at least one lateral upper knife channel wing 228 that extends away from the upper knife channel portion 226. While the term ‘cylindrical’ is used, the channel portion 226 need not resemble a perfect cylinder.

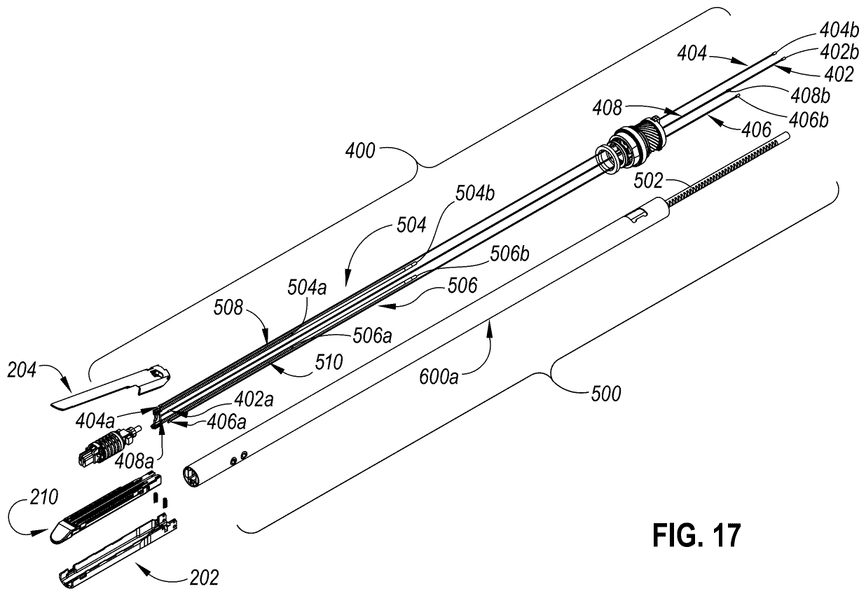

As shown in FIGS. 2 and 17, the surgical instrument 1000 further comprises a knife firing subsystem 500 operable to close the anvil jaw 204 during a closure stroke. After the end effector 200 is closed, the knife firing subsystem 500 is operable to incise and staple, with staples from the staple cartridge 210, the patient tissue captured between the staple cartridge 210 (which is retained by the cartridge jaw 202) and anvil jaw 204 during a firing stroke.

As shown best in FIGS. 4-6, the knife firing subsystem 500, explained further below in greater detail, includes the knife 206 having a knife sled 236. The knife sled 236 functions as a firing driver by driving cartridge sled 210A distally through a firing stroke, as described below. In some instances, knife sled 236 may be referred to as an I-beam. The knife 206 includes a cutting surface 254. The cutting surface 254 is positioned between the first and second lateral wings 241a, 241b.

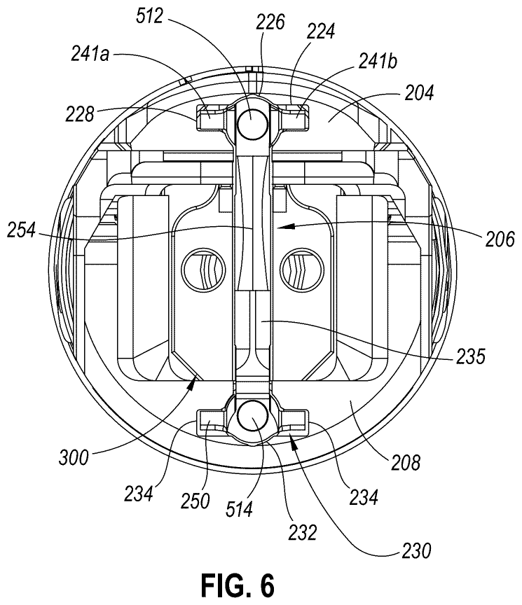

The knife sled 236 includes an upper knife tab 238, a lower knife tab 246, and a vertical column 235 coupling and extending between upper knife tab 238 and lower knife tab 246. The upper knife tab 238 includes a centrally disposed cylindrical upper knife tab portion 240 and at least one upper knife tab lateral wing (shown as first and second lateral wings 241a, 241b) that extend away from the upper knife tab portion 240. While the term ‘cylindrical’ is used, the tab portion need not resemble a perfect cylinder. The knife sled 236 includes at least one lateral wing configured to contact the ramp surface 216. As shown in FIG. 5, the upper knife tab 238 of the knife sled 236 includes first and second lateral wings 241a, 241b configured to contact the ramp surface 216.

The first and second lateral wings 241a, 241b are configured to slidably ride in the upper knife channel 224 to move the anvil jaw 204 between the open position, the grasping position, and the clamping position. Accordingly, the end effector 200 employs “knife-based closure” in which closure of the anvil 204 relative to the channel 202 is driven by distal advancement of the knife 206. Each lateral wing 241a, 241b may include a ramped surface 242A that engages the anvil ramp surface 216. The upper knife tab portion 240 defines an upper knife tab opening 244 that is configured to receive a barrel crimp coupled to a center cable 512, which is described in greater detail below. The lower knife tab 246 includes a centrally disposed cylindrical lower knife tab portion 248 and at least one lower knife tab lateral wing 250 that extends away from the lower knife tab portion 248. While the term ‘cylindrical’ is used, the lower knife tab portion 248 need not resemble a perfect cylinder. In some versions, the lower knife tab 246 includes a pair of lateral wings 250. The lower knife tab portion 248 defines a lower knife tab opening 252 that is configured to receive a barrel crimp coupled to a center cable 514, as described in greater detail below.

The staple cartridge 210 may be generally constructed and operable in accordance with the teachings of U.S. patent application Ser. No. 18/588,684, entitled “Methods of Surgical Stapling,” filed on Feb. 27, 2024, the disclosure of which is incorporated by reference herein in its entirety. In use, the end effector 200 is positioned relative to patient tissue such that the staple cartridge 210 is disposed on a first side of the tissue and the anvil jaw 204 is positioned on an opposed second side of the tissue. The anvil jaw 204 is then approximated toward the staple cartridge 210 to compress and clamp the tissue against the deck of the staple cartridge 210. Thereafter, the surgical instrument 1000 is fired so that the knife 206 advances distally through the staple cartridge 210 to both cut the clamped tissue using cutting surface 254 and simultaneously actuate staple drivers housed within the staple cartridge 210 to drive an array of staples into the clamped tissue on either side of the cut line. Staple cartridge 210 defines an elongate knife channel 215 dimensioned to receive a portion of vertical column 235 in order to accommodate advancement of the knife 206 through staple cartridge 210. A portion of cartridge sled 210A is slidably housed within elongated knife channel 215 such that the vertical column 235 drives the cartridge sled 210A distally as the knife 206 advances distally in accordance with the description herein (see FIGS. 8C-8D). In some instances, the cartridge sled 210A remains in the distal position (see FIG. 8D) relative to the rest of the staple cartridge 210, even after the knife 206 is retracted proximally after firing the staple cartridge 210 in accordance with the description herein.

As mentioned above, cartridge jaw 202 defines an elongated channel 208 for receiving staple cartridge 210. Additionally, cartridge jaw 202 also defines a lower knife channel 230 (see FIGS. 4, 6, and 8A-9D) dimensioned to slidably receive lower knife tab 246. Referring to FIG. 6, the lower knife channel 230 includes a centrally disposed cylindrical lower knife channel portion 232 and at least one lateral lower knife channel wing 234 that extends away from the lower knife channel portion 232. The cylindrical lower knife channel portion 232 is in communication with elongated channel 208 such that when the staple cartridge 210 is suitably coupled to the cartridge jaw 202, the elongated knife channel 215 of staple cartridge 210 and centrally disposed cylindrical lower knife channel portion 232 are aligned to accommodate actuation of knife sled 236 within both channels 215, 230. The lateral lower knife channel wings 234 are dimensioned to slidably house a respective lower knife tab lateral wing 250. Lower knife tab lateral wings 250 are configured to slidably contact the lateral lower knife channel wings 234 as the knife 206 is advanced in accordance with the description herein. Contact between lower knife tab lateral wings 250 and lateral lower knife channel wings 234 cooperatively assists the lateral wings 241a, 241b and the upper knife channel 224 to close the anvil jaw 204 relative to channel 208 in accordance with the description herein. While the term ‘cylindrical’ is used, the channel portion 232 need not resemble a perfect cylinder. Other arrangements of staple cavities and staples may be possible. For example, in some versions, a lower knife channel 230 may be defined in the cartridge jaw 202.

The knife 206 is configured to move relative to the first and second jaws 202, 204. The knife 206 is configured to contact the ramp surface 216 to transition the first and second jaws 202, 204 from an open position (see FIG. 8A) to a closed position (see FIG. 8C). As shown in FIGS. 9A-9D, the knife 206 is configured to pivot the second jaw 204 relative to the first jaw 202 as the knife 206 moves distally along the ramp surface 216.

Further to the above, the knife sled 236 is moved distally and proximally by a firing rod 502. The firing rod 502 is configured to apply an indirect force to the knife sled 236, via push coils 508, 510 that directly engage the knife sled 236 (discussed in greater detail below), and push the knife sled 236 toward the distal end of the end effector 200 through a firing stroke. As the firing rod 502 is advanced distally, the knife sled 236 rides in the lower knife channel 230 and the upper knife channel 224. At the onset of travel, the upper knife tab 238 rides along the anvil ramp surface 216. Specifically, as particularly seen in the sequence of FIGS. 8A-8D and 9A-9D, movement of the knife sled 236 distally causes the upper knife tab ramped surface 242A to slide along the anvil ramp surface 216. This movement first forces the anvil jaw 204 to close to a position (e.g., FIGS. 8B and 9B) where a compressive force is applied to the tissue sufficient to grasp it and prevent it from slipping during firing (referred to as the grasping position). Continued movement of the knife sled 236 up the ramp surface 216 (see e.g., FIGS. 8C and 9C) results in a compressive force being applied to the tissue (referred to as the clamping position). As the anvil ramp surface 216 transitions to its substantially horizontally angled surface 220 (see FIGS. 8D and 9D), the upper knife tab 238 can slide within the upper knife channel 224 to drive the stapling and transection of the tissue.

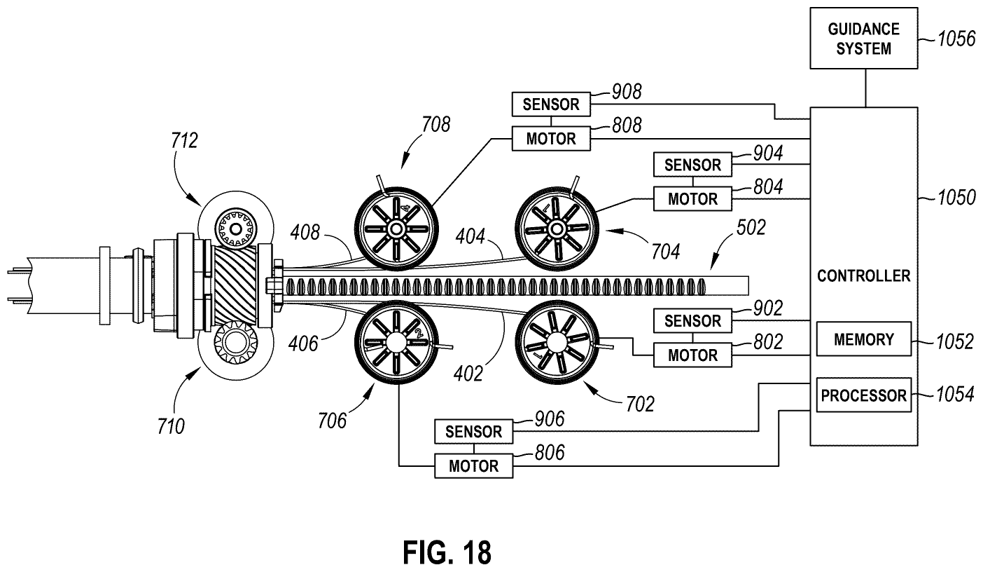

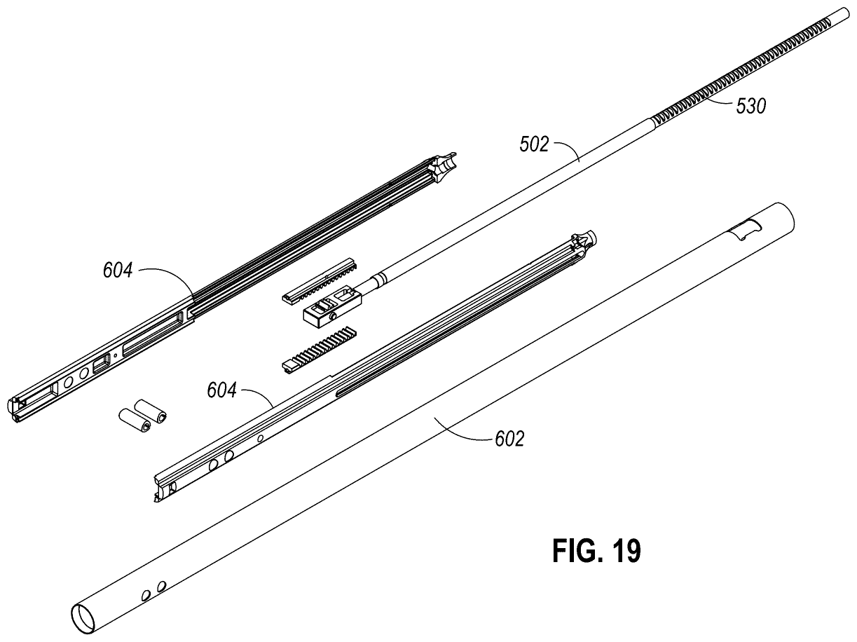

As shown in FIG. 1, the surgical instrument 1000 further comprises a body exemplified as a housing 700 configured to engage a robotic platform (not shown). In other versions, the body may be configured as a handle (not shown) configured to be gripped and manipulated by a clinician. As best shown in FIGS. 1 and 19, a shaft assembly 600A extends distally from the housing 700 and includes a rotatable outer shaft 602 and an inner shaft 604 arranged in two clamshell halves, with the outer shaft 602 being rotatably mounted to the housing 700 about a rotation joint (not shown), which may include one or more bearings. The inner shaft 604 is rotationally fixed to the outer shaft 602 and is configured such that articulation cables 402, 404, 406, 408 can be partially wound therearound without becoming tangled. As shown in FIG. 18, the housing 700 may house (1) a firing puck assembly 712 (as part of the knife firing subsystem 500 (see FIG. 17)) operable to close the end effector 200, fire staples, and transect tissue, (2) a set of articulation puck assemblies 702, 704, 706, 708 as part of the articulation subsystem 400 operable to articulate the end effector 200 relative to the shaft assembly 600A, and (3) a shaft roll puck assembly 710 as part of the roll subsystem 600 configured to roll the outer shaft 602. In other words, the firing puck assembly 712 connects the motor 1100 to the knife firing subsystem 500, which is used to open/close the end effector 200, grasp/clamp on tissue, transect tissue, and fire staples.

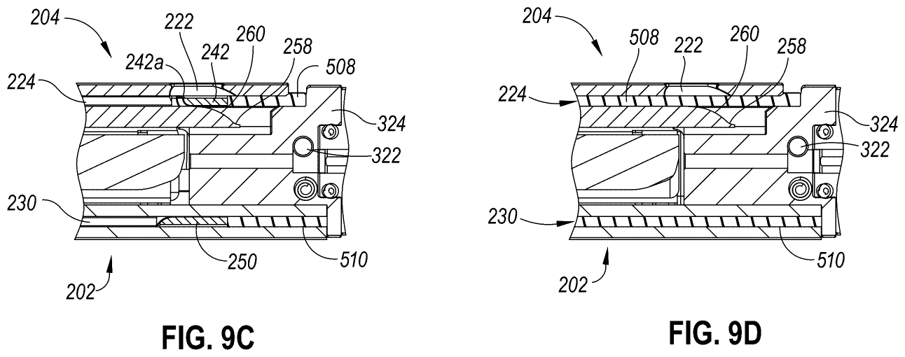

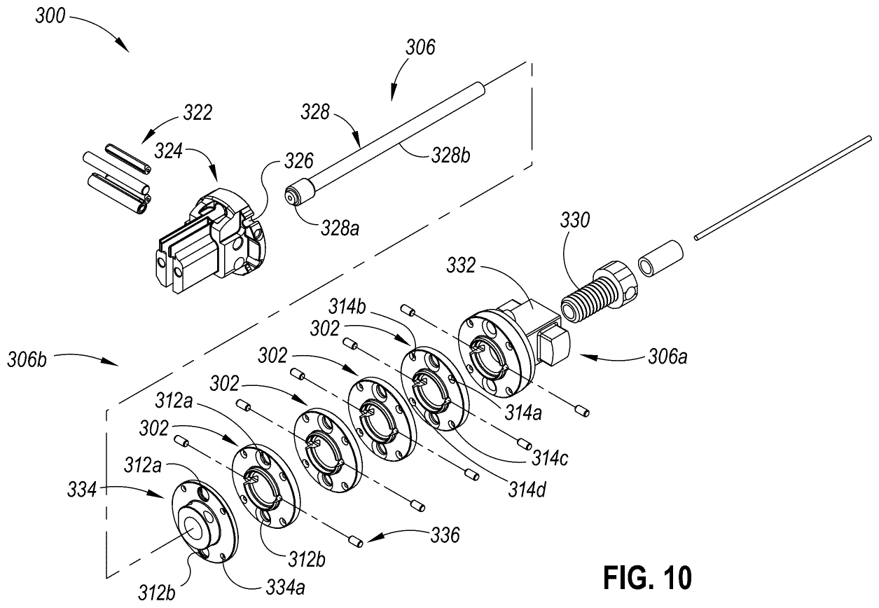

Referring to FIGS. 10-13, the articulation joint 300 comprises an array of joint discs 302 arranged longitudinally, and a center lumen assembly 306 that cooperates with the joint discs 302 to provide articulation of the end effector 200 with at least two degrees of freedom (e.g., yaw and pitch), as described further below. Each joint disc 302 includes a central opening 304 that is configured to align coaxially with the central opening 304 of the other joint discs when the articulation joint 300 is in a straight, non-articulated state. The center lumen assembly 306 extends longitudinally through the central openings 304 of joint discs 302 and applies a compressive axial force to the array of joints discs 302 to couple the joint discs 302 with one another. The joint discs 302 are nestably stacked with one another along the center lumen assembly 306 such that longitudinally adjacent joint discs 302 movably interface with one another.

As seen in FIGS. 9A-10, a distal end 306B of the center lumen assembly 306 includes a distal retainer 324 that couples the distal end of the articulation joint 300 with a proximal end of the cartridge jaw 202 via one or more fasteners 322, thereby mechanically grounding and retaining the cartridge jaw 202 and thus the end effector 200 relative to the articulation joint 300. The distal retainer 324 includes a plurality of clearance pockets 326 that receive distal ends of articulation cables 402, 404, 406, 408. The distal end 306B further includes a distal retention disc 334 that defines a plurality of cable retention openings 334A. A proximal end 306A of the center lumen assembly 306 includes a proximal retainer 332 that couples the proximal end of the articulation joint 300 with a distal end of the shaft assembly 600A.

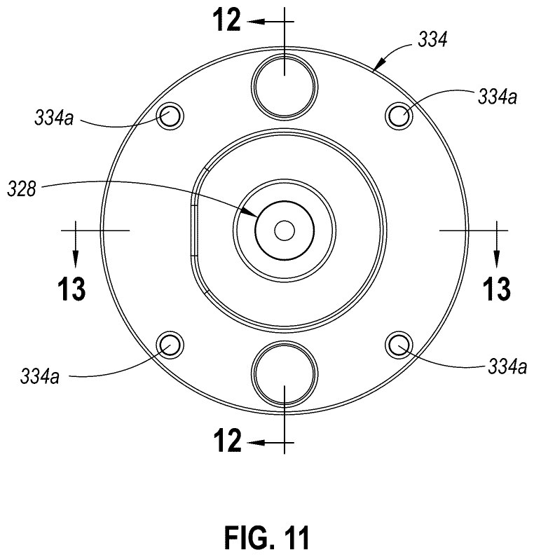

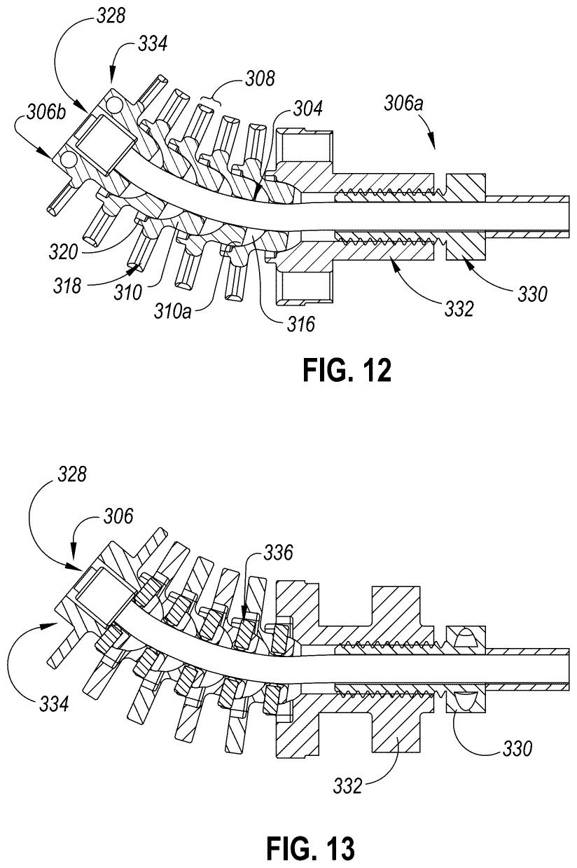

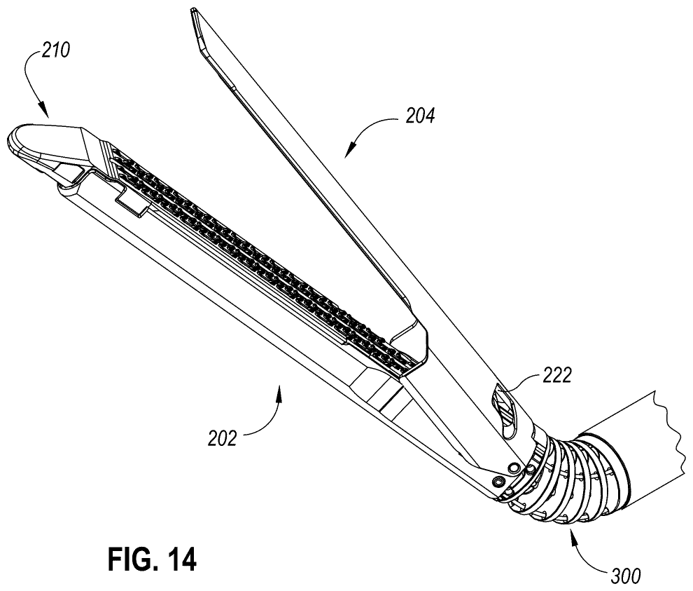

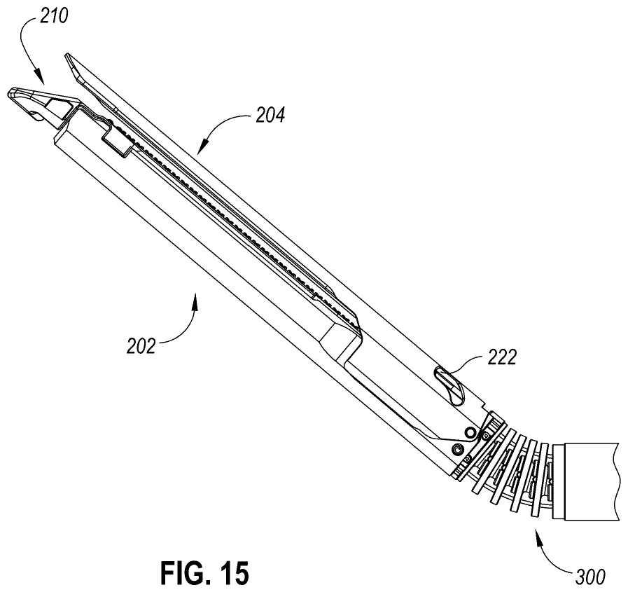

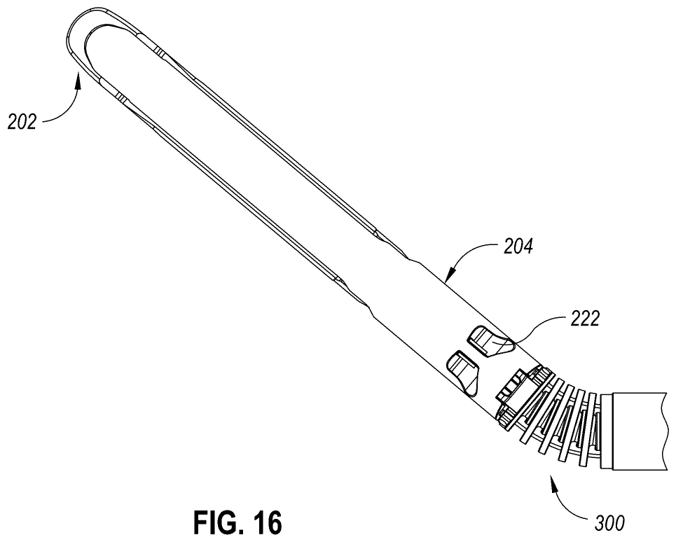

As shown particularly in FIGS. 10, 12, and 13, each joint disc 302 includes an articulation socket 308, an articulation pin 310 protruding outwardly from the articulation socket 308, a first push coil opening 312A defined through the articulation socket 308 and configured to receive a first push coil 508 therethrough, a second push coil opening 312B defined through the articulation socket 308 and configured to receive a second push coil 510 therethrough, and a plurality of articulation cable openings 314A-314D (e.g., a first articulation cable opening 314A, a second articulation cable opening 314B, a third articulation cable opening 314C, and a fourth articulation cable opening 314D) defined through the articulation socket 308 and configured to receive a respective articulation cable 402, 404, 406, 408 (e.g., a first articulation cable 402, a second articulation cable 404, a third articulation cable 406, and a fourth articulation cable 408) therethrough, and discussed in greater detail below. As shown in FIGS. 12 and 13, the central opening 304 is defined in the articulation pin 310 of each joint disc 302. In some versions, three articulation cable openings 314A, 314B, 314C are provided to correspond to three articulation cables 402, 404, 406, while in other versions, four articulation cable openings 314A, 314B, 314C, 314D are provided to correspond to four articulation cables 402, 404, 406, 408.

Each joint disc 302 further includes a rounded articulation pin proximal end 310A and a semi-spherical pin-receiving opening 316 defined in the articulation socket 308. As shown particularly in FIGS. 12 and 13, each rounded articulation pin proximal end 310A pivotally engages in an adjacent pin-receiving opening 316 of an adjacent joint disc 302, with the exception of a 332d 310A that engages with the proximal retainer 332. The articulation pin proximal end 310A and pin-receiving opening 316 interface functions in a similar manner as a swivel bearing. Moreover, the articulation socket 308 includes a socket disc 318 and a pin retention socket 320. A pair of pins 336 are used to provide rotational coupling about the roll axis of the shaft assembly 600A from one joint disc 302 to the next. In other words, the pins constrain a rotational degree of freedom between adjacent joint discs 302 about the roll axis RA of the surgical instrument 1000. In alternative versions, this feature can be integral to the joint disc 302.

The center lumen assembly 306 further includes a center lumen 328 that extends longitudinally through the central openings 304 of the joint discs 302. The center lumen 328 includes a nitinol core 328A and a stainless-steel collar 328B wound over the nitinol core 328A that allows the center lumen 328 to resiliently flex during deflection of the articulation joint 300. The wound stainless-steel collar 328B may have clockwise braiding and counterclockwise braiding to prevent unwinding thereof. The center lumen assembly further includes a jack screw 330 that is threadably coupled with the proximal retainer 332 to adjust an axial compression force exerted by the center lumen 328 on the array of joint discs 302, thereby enabling adjustment of a pre-load of the articulation joint 300.

The above-described articulation joint 300 forms a portion of the cable articulation subsystem 400 which allows for precise 360-degree articulation of the end effector 200 about the articulation joint 300 with at least two degrees of freedom. In some versions, and as dictated by the roll subsystem 600 as well as a need to limit the amount of wrap of the articulation cables 402, 404, 406, 408, the articulation joint 300 is permitted about 320 degrees of roll in both a clockwise and counterclockwise direction within the overall system. The cable articulation subsystem 400 also includes a plurality of articulation cables 402, 404, 406, 408 each having a distal end 402A, 404A, 406A, 408A, coupled to the distal end 306B of the center lumen assembly 306, and a proximal end 402B, 404B, 406B, 408B. More specifically, each distal end 402A, 404A, 406A, 408A can include a crimp that engages a cable retention opening 334A of the distal retention disc 334 to maintain its positioning. Each articulation cable is discretely manipulable to cause rotation of the articulation joint 300 and end effector 200 about at least one of a pitch axis PA and a yaw axis YA.

In some versions, three articulation cables may be provided rather than the four cables 402, 404, 406, 408 depicted herein. However, four articulation cables 402, 404, 406, 408 circumferentially spaced approximately ninety degrees from one another (as shown) provide load splitting over the full 360 degree range of conical workspace of the wrist. Additionally, in alternative versions, the articulation cable configuration may be non-symmetric.

The shaft assembly 600A and housing 700 also form portions of the cable articulation subsystem 400. More specifically, each articulation cable 402, 404, 406, 408 extends from the articulation joint 300 and through the shaft assembly 600A to the housing 700. The proximal end 402B, 404B, 406B, 408B of each articulation cable (402, 404, 406) is movably mounted in the housing 700 which causes the above-mentioned rotation of the articulation joint 300 and end effector 200. The housing 700 includes articulation puck assemblies 702, 704, 706, 708 with rotatable capstans (not shown) about which corresponding proximal ends 402B, 404B, 406B, 408B of the articulation cables 402, 404, 406, 408 are spooled and unspooled.

The articulation cables 402, 404, 406, 408 are routed through the shaft assembly 600A such that they are disposed between the outer shaft 602 and the inner shaft 604, with the articulation cables 402, 404, 406, 408 being able to partially spooled therearound without becoming tangled. The inner shaft 604 also prevents the articulation cables 402, 404, 406, 408 from interfering with other components running down the center of the surgical instrument 1000 (through the inner shaft 604).

The articulation cables 402, 404, 406, 408 are routed and coupled to the end effector 200 via the articulation joint 300 such that movement thereof in a proximal direction (via winding about the capstans of the housing 700) causes the end effector 200 to articulate in a predetermined manner via the articulation joint 300. For example, actuation of the first articulation cable 402 in the proximal direction causes articulation of the end effector 200 upwards and to the left, actuation of the second articulation cable 404 in the proximal direction causes rotation of the end effector 200 upwards and to the right, actuation of the third articulation cable 406 in the proximal direction causes rotation of the end effector 200 downwards and to the left, and actuation of the fourth articulation cable 408 in the proximal direction causes rotation of the end effector 200 downwards and to the right. Similarly, movement of two or more of the articulation cables simultaneously will result in compound motion (e.g., articulation) of the end effector 200. As will be appreciated by those skilled in the art, this configuration provides for the above-mentioned precise 360-degree articulation of the end effector 200 via the articulation joint 300 with at least two degrees of freedom and about at least 320 degrees of roll in both a clockwise and counterclockwise direction.

Referring to FIG. 18, a plurality of motors 802, 804, 806, 808 can be associated with the articulation puck assemblies 702, 704, 706, 708 to facilitate rotation of each of the capstans to thereby cause the corresponding articulation cables 402, 404, 406, 408 to extend or retract (depending on the direction of rotation). The puck assemblies 702, 704, 706, 708 can accordingly facilitate operable coupling of the motors 802, 804, 806, 808 to the articulation cables 402, 404, 406, 408. Each of the motors 802, 804, 806, 808 can be operated independently to change the desired articulation position (e.g., the pose) of the end effector 200 through manipulation of articulation cables 402, 404, 406, 408, as described above.

The motors 802, 804, 806, 808 can be in communication with a controller 1050. The controller 1050 can include a memory 1052 and a processor 1054. A guidance system 1056 can be in communication with the controller 1050 that is configured to provide user control of the motors 802, 804, 806, 808 via the controller 1050 of the desired pose of the end effector 200. In some instances, the guidance system 1056 can be provided onboard the surgical instrument 1000 in the form of buttons, a joystick, or other user actuated controls that allows a surgeon to select the desired pose of the end effector 200 from the surgical instrument. In other instances, the guidance system 1056 can be remote from the surgical instrument 1000 as a stand-alone control device or as part of a robotic surgical system.

The motors 802, 804, 806, 808 can be any of a variety of rotary motors that are capable of rotating the capstans independently in different directions. The motors 802, 804, 806, 808 can have indexing capabilities that allows each of the motors 802, 804, 806, 808 to be rotated to a specific position as well as the angular position of each of the motors 802, 804, 806, 808 to be detected by the controller 1050. As such, the tension on the articulation cables 402, 404, 406, 408 can be controlled through selective indexing of the motors 802, 804, 806, 808. Some examples of these types of motors include a stepper motor, an encoder-type motor, a brushed or brushless DC motor, or a servo motor. It is to be appreciated that the articulation cables 402, 404, 406, 408 can be associated with any of a variety of suitable alternative drive systems for actuation thereof. In one alternative example, the articulation cables 402, 404, 406, 408 can be associated with a plurality of linear motors to extend and retract the articulation cables 402, 404, 406, 408 via a plunger or suitable alternative component. The linear motors can be indexable such that the plunger can be moved to a specific position (e.g., to control the tension on the articulation cables 402, 404, 406, 408) that is detectable by the controller 1050.

A plurality of sensors 902, 904, 906, 908 can be associated with the motors 802, 804, 806, 808 to facilitate sensing of the tension on the articulation cables 402, 404, 406, 408. The sensors 902, 904, 906, 908 can be in communication with the controller 1050. In some versions, each of the sensors 902, 904, 906, 908 can comprise a torque sensor configured to sense torque of the respective motors 802, 804, 806, 808. The controller 1050 can be configured to extrapolate the tension exerted on each of the 402, 404, 406, 408 (e.g., a tension value) as a function of the sensed torque from the sensors 902, 904, 906, 908. In an alternative example, the sensors 902, 904, 906, 908 can comprise a tension sensor that is associated directly with the articulation cables 402, 404, 406, 408 and is configured to report the sensed tension directly to the controller 1050. The motors 802, 804, 806, 808, the sensors 902, 904, 906, 908, and the controller 1050 can be sized and configured to be positioned within the housing 700.

As shown throughout FIGS. 2, 4, 5, 8A-8D, 9A-9D, 17 and 19, the knife firing subsystem 500 includes the aforementioned knife 206, the aforementioned knife sled 236, a firing rod 502 that drives the knife 206 and/or knife sled 236, a first push rod 504, and a second push rod 506. The firing rod 502 includes a firing rack 530 and is driven by a firing puck assembly 712 of the housing 700 which is operatively coupled with the motor 1100. The first push rod 504 has a first push rod distal end 504A coupled to push coil 508 and a first push rod proximal end 504B coupled to the firing rod 502. Similarly, the second push rod has a second push rod distal end 506A coupled to push coil 510 and a second push rod proximal end 506B coupled to the firing rod 502. The distal ends of push coils 508, 510 are coupled to respective upper and lower portions of the knife sled 236 (e.g., the upper knife tab 238 and the lower knife tab 246), which enables the knife 206 to be pushed evenly at its ends. In some versions, the proximal ends 504B, 506B of the push rods 504, 506 are coupled to the firing rod 502 via a linear differential 520.

The knife firing subsystem 500 is configured in a manner to enable articulation of the end effector 200 while still enabling proper functionality of the knife 206. To that end, the first push rod 504 includes a first flexible section in the form of a first push coil 508 and the second push rod 506 comprises a second flexible section in the form of a second push coil 510. The push coils 508, 510 route through the articulation joint 300 via the respective push coil openings 312A, 312B, and the push rods 504, 506 engage the respective tab openings 244, 252 in the knife sled 236. A first center cable 512 extends through the first push coil 508 to engage the knife sled 236 via a barrel crimp, and a second center cable 514 extends through the second push coil 510 to engage the knife sled 236 via a barrel crimp. The push coils 508, 510 provide the push rods 504, 506 sufficient column strength to deliver an axial firing force to the knife 206, while not being too stiff that would prevent articulation at the joint 300. The cables 512, 514, which are engaged with the knife sled 236 as discussed above (see, e.g., FIG. 8A), prevent the push coils 508, 510 from stretching and/or elongating and serve as retraction cables when the rods 504, 506 are retracted towards the proximal end of the surgical instrument 1000. The entirety of each push rod 504, 506 does not extend through the articulation joint 300, and therefore does not need to be flexible. Accordingly, a proximal section of each push rod 504, 506 can be less flexible than the push coils 508, 510.

### II. Controlling Tension in Articulation Cables of Surgical Stapler

As described above, the end effector 200 can be articulated into a desired pose to better position the end effector 200 for interacting with a tissue during a surgical procedure. The articulation of the end effector 200 can be achieved by selectively and independently operating motors 802, 804, 806, 808 to extend and retract the articulation cables 402, 404, 406, 408. The resulting tensions that are exerted onto the articulation cables 402, 404, 406, 408 can be within the normal operating tolerances of the articulation cables 402, 404, 406, 408 and the articulation joint 300 (e.g., within the tension budget). However, when tissue is clamped between the jaws 182, 184 and the knife 206 is fired, the force from one or both of those actions can exert excessive tension on the articulation cables 402, 404, 406, 408 that could exceed the normal operating tolerances of the articulation cables 402, 404, 406, 408. This excessive tension can fatigue the articulation cables 402, 404, 406, 408, motors 802, 804, 806, 808, and/or puck assemblies 702, 704, 706, 708 over time which can lead to reduced accuracy when achieving a desired pose, inconsistent cutting performance, and in some cases, component failure. As a result, it may be beneficial for the controller 1050 to regulate the tension exerted on the cables during clamping and/or firing to prevent excessive tension from being exerted on the cables. It may also be beneficial for the controller 1050 to regulate the tension in such a way that still maintains the end effector 200 in the desired pose that was originally selected during initialization of the surgical procedure.

### A. Method of Reducing Tensions on Articulation Cables to a Threshold

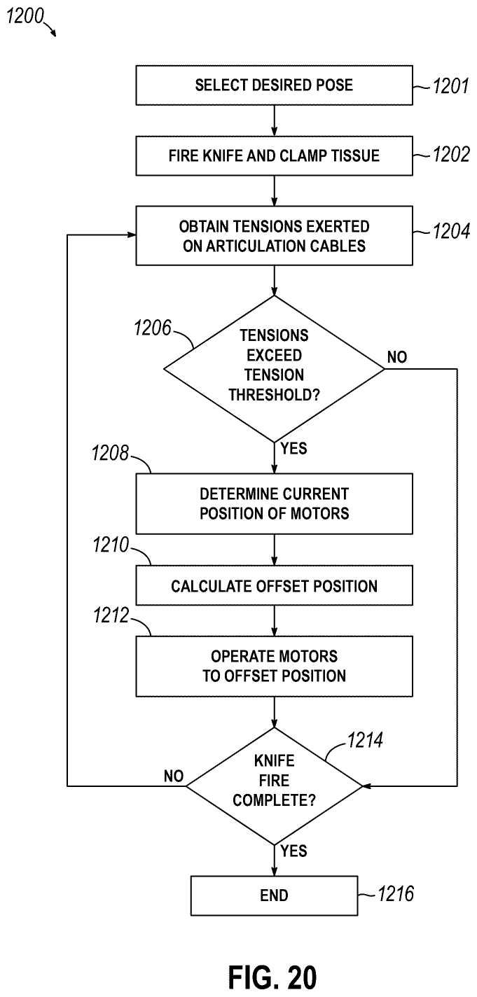

A method 1200 of operating the surgical instrument 1000 is illustrated and described with reference to FIG. 20. The method 1200 uses tension feedback from the sensors 902, 904, 906, 908 to maintain the tension below a predefined threshold in real time. The method 1200 also can facilitate regulation of the tension on the articulation cables 402, 404, 406, 408 in such a manner that prevents inadvertent repositioning of the desired pose when the tension on the articulation cables 402, 404, 406, 408 is changed.

At step 1201, a user selects the desired pose of the end effector 200. At step 1202, the user or the controller 1150 initiates clamping of the tissue and firing of the knife 206 through the tissue which may be automatically or manually initiated. In some arrangements, the clamping of the tissue can occur in response to the firing of the knife being initialized, as described above. In other arrangements, the clamping of the tissue and the firing of the knife can occur as separate operations such that the clamping of the tissue is a condition precedent to firing of the knife 206.

Once the knife 206 begins to fire, the controller 1050 can obtain the tension exerted on each of the articulation cables 402, 404, 406, 408 from the sensors 902, 904, 906, 908, at step 1204, and can then compare the tensions to a tension threshold to determine if the tensions exceed the tension threshold, at step 1206. In one example, the magnitude of each of the respective tensions exerted on the articulation cables 402, 404, 406, 408 can be compared against the tension threshold. Alternatively, an average of the tensions can also be compared against the tension threshold.

The tension threshold can be selected to prevent the tension levels on the articulation cables 402, 404, 406, 408 from reaching a level that might adversely affect the operation of the end effector 200 and/or the surgical procedure being conducted. In some examples, the tension threshold can take into account the operating limits of the electrosurgical instrument 1000 (e.g., the tension limits of the articulation cables 402, 404, 406, 408 and/or the capabilities of the motors 802, 804, 806, 808 to produce certain tensions) as well as the necessary tensions that must be maintained to ensure the integrity of the surgical procedure being conducted by the electrosurgical instrument 1000. The tension threshold can be predefined and stored in the memory 1052 of the controller 1050 for reference or alternatively can be determined in real time (e.g., as a function of the current operation conditions of the end effector 200 and/or the surgical instrument 1000).

If any of the tensions exerted on the articulation cables 402, 404, 406, 408 are above the tension threshold, the controller 1050 can determine the current positions of the motors 802, 804, 806, 808, at step 1208, and can calculate an amount that each of the motors 802, 804, 806, 808 should be moved (hereinafter referred to as an offset position), at step 1210, to unwind (e.g., payout) the articulation cables 402, 404, 406, 408 enough to reduce the tensions below the tension threshold. The controller 1050 can then move the motors 802, 804, 806, 808 to the offset position, at step 1212. Each of the motors 802, 804, 806, 808 can be moved in unison and the same amount to prevent the resulting movement of the articulation cables 402, 404, 406, 408 from changing the desired pose of the end effector 200. In other words, the articulation cables 402, 404, 406, 408 can be extended collectively and equally to substantially maintain the position of the end effector 200 with respect to the shaft assembly 600. It is to be appreciated that describing the position of the end effector as being substantially maintained can be understood to mean that to the extent the position of the end effector 200 is changed, such change is miniscule enough that it does not affect the surgical procedure that is being conducted. In some examples, that can mean that the position of the end effector 200 is maintained within a predefined threshold range of about +−5% of the overall articulation range of the end effector 220.

Once the motors 802, 804, 806, 808 have been moved to the offset position, the controller 1050 can then determine, at step 1214, whether the firing of the knife has been completed. Similarly, if the tensions exerted on the articulation cables 402, 404, 406, 408 are determined to be below the tension threshold in step 1206, steps 1208, 1210, 1212 can be skipped and the method 1200 can proceed directly to step 1214. In either scenario, if the firing of the knife is not complete, the tensions of the articulation cables 402, 404, 406, 408 can continue to be reevaluated and adjusted via steps 1208, 1210, 1212, to ensure that the tensions remain at or below the tension threshold until the firing of the knife firing is complete, at step 1216. It is to be appreciated that although method 1200 is described as monitoring and adjusting the tensions of the articulation cables 402, 404, 406, 408 during firing of the knife 206, the method 1200 can be utilized for any of a variety of other processes during a surgical procedure where regulating the tensions on the articulation cables 402, 404, 406, 408 is desired, such as, for example, during clamping of the tissue. The methods for regulating the tensions on the articulation cables 402, 404, 406, 408 during these processes can be conducted in addition to, or alternative to, the firing of the knife 206.

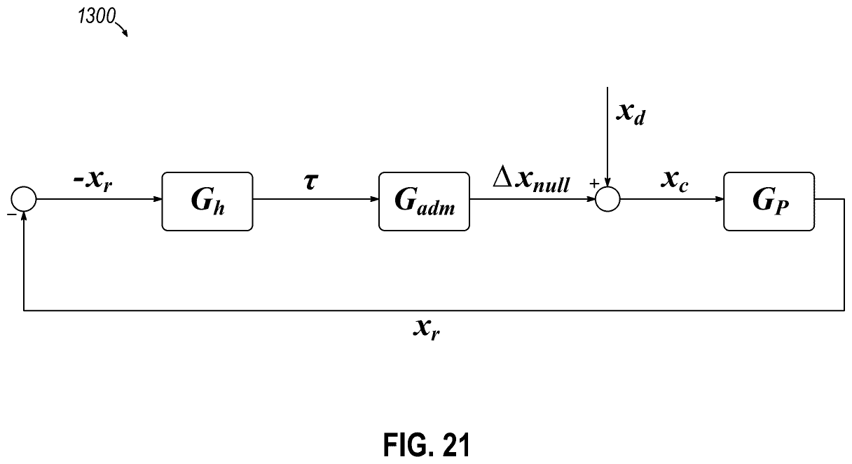

FIG. 21 illustrates a block diagram of an architecture of a feedback control system 1300 that embodies the method 1200 and that can be implemented by the controller 1050 when executing the method 1200. Δ_{Xnull} can represent the offset position, τ can represent detected tension, X_{c} can represent the command that is sent to the motors 802, 804, 806, 808 for positioning the motors, Xᵣ can represent a reference position and X_{d} can represent an initial position of the motors 802, 804, 806, 808 that achieves the desired pose. A closed loop position controller Gₚ can be implemented at the motor level and can control positioning of the motors 802, 804, 806, 808 in response to the command X_{c}. A model Gₕ can be provided that represents a plant model that relates a reference position of the motors to a motor torque, and an admittance filter G_{adm} can be provided that is responsible for converting torque/force data from the motors/cables to a position offset. The admittance filter G_{adm} operates based on the tension threshold and the amount by which the current tension value exceeds the threshold.

During firing of the knife 206, the detected tensions t can be provided to the admittance filter G_{adm}. The admittance filter G_{adm} can compare the detected tensions t to the threshold tension and can generate the offset position Δ_{Xnull} for the motors 402, 404, 406, 408. If the detected tensions τ do not exceed the tension threshold, the offset position Δ_{Xnull} is zero. If the detected tensions τ exceed the tension threshold, the offset position Δ_{Xnull} is proportional to the difference between the detected tensions τ and the given tension threshold. In any event, the offset position Δ_{Xnull} is summed with the initial position Xd to generate the command X_{c} for moving the motors 802, 804, 806, 808 by the amount dictated by the offset position Δ_{Xnull} relative to the initial position X_{d}, to reduce the tensions τ on the articulation cables 402, 404, 406, 408 accordingly. In response, the closed loop position controller Gₚ can facilitate movement of the motors 802, 804, 806, 808 accordingly and can indicate the current state of the motors 802, 804, 806, 808 with the reference position Xᵣ. The model Gₕ can then control the responsiveness of the system to any subsequent changes to the tensions and can output the tension value τ to the admittance filter G_{adm} accordingly. In one example, the model Gₕ can be a spring damper model whereby the “mass” and “spring constant” of the model can be selected to control the responsiveness of the system to changes in the tensions. This process can be repeated throughout firing of the knife 206 to regulate the tensions on the articulation cables 402, 404, 406, 408 accordingly.

In an alternative example, each of the articulation cables 402, 404, 406, 408 can be controlled independently to selectively reduce one or more of the tensions exerted thereon when those tensions exceed the tension threshold. In such an example, steps 1202, 1204, 1206, 1208, 1210, and 1212 can be performed separately on each individual articulation cable 402, 404, 406, 408 to reduce any of the tensions that exceed the tension threshold to at or below the tension threshold. The selective reduction of the tensions of the articulation cable 402 relative to the threshold level will now be described in terms of method 1200 but can be understood to be representative of the reduction of the tensions of the other articulation cables 404, 406, 408 relative to the threshold level.

Once the knife 206 begins to fire, the controller 1050 can obtain the tension exerted the articulation cable 402 from the sensors 902, at step 1204, and can then compare the tension with the tension threshold to determine if the tension exceeds the tension threshold, at step 1206. If the tension exerted on the articulation cable 402 is above the tension threshold, the controller 1050 can determine the current position of the motor 802, at step 1208, and can calculate the offset position for the motor 802, at step 1210. The controller 1050 can then move the motor 802 to the offset position, at step 1212. This same process can be employed for each of the other the articulation cables 404, 406, 408 at substantially the same time as the articulation cable 402.

Each of the motors 802, 804, 806, 808 can be operated at the same time according to the respective offset positions that are calculated for each of the motors 802, 804, 806, 808. However, because the tension values might be different among the articulation cables 402, 404, 406, 408, the motors 802, 804, 806, 808 might be moved to different positions relative to each other. In addition, even though some of the tensions on the articulation cables 402, 404, 406, 408 might not exceed the tension threshold, the tensions on those articulation cables 402, 404, 406, 408 might still be reduced to allow all of the tensions to be reduced in such a manner as to substantially maintain the position of the end effector 200 with respect to the shaft assembly 600. As such, the controller 1050 can be configured to coordinate and regulate the operation of the motors 802, 804, 806, 808 in order to substantially maintain the pose of the end effector 200 with respect to the shaft assembly 600 when reducing the tension exerted on at least one of the articulation cables 402, 404, 406, 408 to at or below the tension threshold.

### B. Method of Regulating Tensions on Articulation Cables to an Upper Threshold and a Lower Threshold

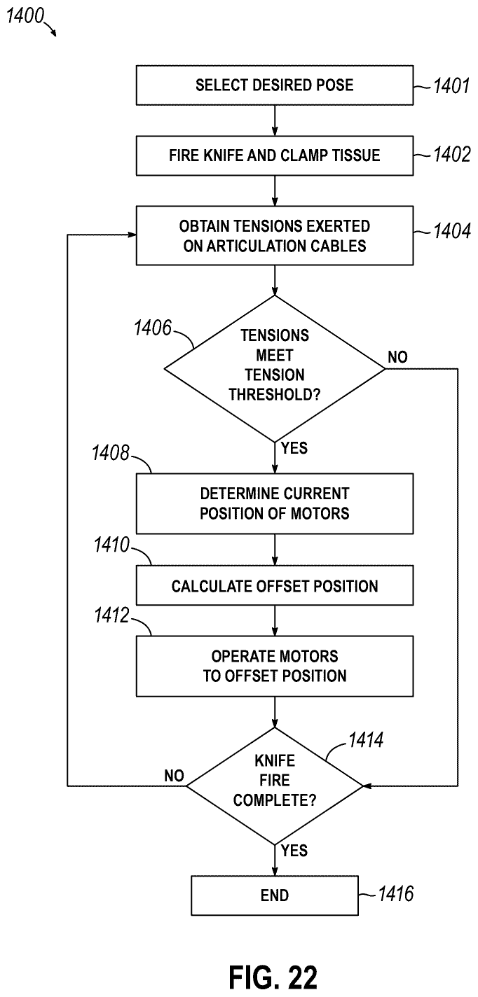

A method 1400 of operating the surgical instrument 1000 is illustrated and described with reference to FIG. 22 and can be understood to be similar to, or the same in many respects as, the method 1200. However, method 1400 uses tension feedback from the sensors 902, 904, 906, 908 to regulate the tensions on the articulation cables 402, 404, 406, 408 towards a predefined threshold value or range of values in real time. The method 1400 also can facilitate regulation of the tension on the articulation cables 402, 404, 406, 408 in such a manner that prevents inadvertent repositioning of the desired pose when the tension on the articulation cables 402, 404, 406, 408 is changed.

At step 1401, a user selects the desired pose of the end effector 200. At step 1402, the user or the controller 1050 initiates clamping of the tissue and firing of the knife 206 through the tissue which may be automatically or manually initiated. Once the knife 206 begins to fire, the controller 1050 can obtain the tensions exerted on each of the articulation cables 402, 404, 406, 408 from the sensors 902, 904, 906, 908, at step 1404, and can then compare the tensions with an upper and a lower tension threshold to determine if the tensions exceed or fall below the upper and lower tension thresholds, respectively, at step 1406. If any of the tensions exerted on the articulation cables 402, 404, 406, 408 exceed or fall below the upper and lower tension thresholds, respectively, the controller 1050 can determine the current positions of the motors 802, 804, 806, 808, at step 1408, and can calculate the offset position for each of the motors 802, 804, 806, 808 that should be employed, at step 1410, to either wind or unwind the articulation cables 402, 404, 406, 408 enough to increase or reduce the tensions, respectively, such that they no longer exceed or fall below the upper and lower tension thresholds, respectively. The controller 1050 can then move the motors 802, 804, 806, 808 to the offset position, at step 1412 and can control/coordinate their operation such that the end effector 200 substantially maintains its pose with respect to the shaft assembly 600.

Once the motors 802, 804, 806, 808 have been moved to the offset position, the controller 1050 can then determine, at step 1414, whether the firing of the knife has been completed. Similarly, if the tensions exerted on the articulation cables 402, 404, 406, 408 are determined to be below the tension threshold in step 1406, steps 1408, 1410, 1412 can be skipped and the method 1400 can proceed directly to step 1414. In either scenario, if the firing of the knife is not complete, the tensions of the articulation cables 402, 404, 406, 408 can continue to be reevaluated and adjusted via steps 1408, 1410, 1412, to ensure that the tensions are within the upper and lower tension thresholds until the firing of the knife firing is complete, at step 1416.

In some instances, the upper and lower tension thresholds can be different such that upper and lower thresholds define a threshold range which the tensions are maintained within. In other instances, the upper and lower tension thresholds are substantially the same such that the tensions of the articulation cables 402, 404, 406, 408 are substantially maintained at a particular magnitude. In some instances, each of the motors 802, 804, 806, 808 can be moved in unison and the same amount to prevent the resulting movement of the articulation cables 402, 404, 406, 408 from changing the desired pose of the end effector 200. In other instances, each of the articulation cables 402, 404, 406, 408 can be controlled independently to reduce the tensions exerted thereon when any of the tensions exceed the tension threshold.

### C. Method of Tensioning Articulation Cables Prior to Conducting Surgical Procedure

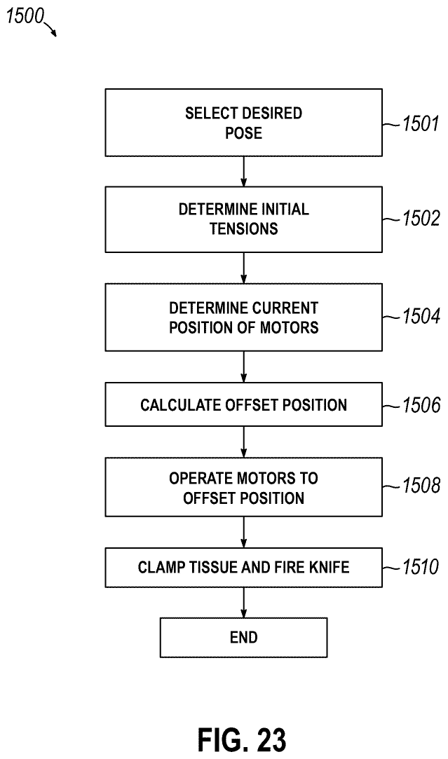

A method 1500 of operating the surgical instrument 1000 is illustrated and described with reference to FIG. 23. The method 1500 utilizes a feedforward algorithm that predicts initial tensions that can be applied to the articulation cables 402, 404, 406, 408 after the pose of the end effector 200 has been selected but prior to firing the knife 206. The initial tensions compensate for the anticipated additional tension on the articulation cables 402, 404, 406, 408 that is likely to occur during a surgical procedure and imparts the initial tension value on the articulation cables 402, 404, 406, 408 accordingly.

At step 1501, a user selects the desired pose of the end effector 200. At step 1502, initial tensions for the articulation cables 402, 404, 406, 408 are calculated that compensates for the anticipated additional tension on the articulation cables 402, 404, 406, 408 that is likely to occur during a surgical procedure. The initial tension value can take into account any physical or operational parameters that are unique to the electrosurgical instrument 1000 that might cause an increase in the tensions on the articulation cables 402, 404, 406, 408 during firing of the knife 206 such as, for example, start time, end time, cable loads, knife load, pose of the end effector, knife velocity, knife mechanism compliance, tissue thickness, and cable stiffness.

Once the initial tensions are calculated, the controller 1050 can determine the current tensions that are exerted on the articulation cables 402, 404, 406, 408, at step 1502. The controller 1050 can then determine the positions of the motors 802, 804, 806, 808, at step 1504, and can calculate the offset position, at step 1506, for each of the motors 802, 804, 806, 808 that reduces the tensions to the initial tension value. The controller 1050 can then move the motors 802, 804, 806, 808 to the offset position, at step 1508 and can control/coordinate their operation such that the end effector 200 substantially maintains its pose with respect to the shaft assembly 600. At step 1510, the user or the controller 1050 initiates clamping of the tissue and firing of the knife 206 through the tissue. In some arrangements, the clamping of the tissue can occur before the method 1500 is initialized such that each of steps 1501, 1502, 1504, 1506, and 1508 occur while the tissue is clamped but prior to the firing of the knife 206, at step 1510.

### III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method for performing an electrosurgical procedure using an electrosurgical instrument (1000), wherein the electrosurgical instrument (1000) includes a shaft (600A), an articulation joint (300) coupled with the shaft (600A), an end effector (200) coupled with the articulation joint (300), a plurality of articulation cables (402, 404, 406, 408) coupled with the end effector (200) via the articulation joint (300) and manipulable to cause the end effector (200) to articulate at the articulation joint (300) with respect to the shaft (600A), a plurality of motors (802, 804, 806, 808) operably coupled with the plurality of articulation cables (402, 404, 406, 408) and configured to articulate the end effector (200) via the articulation cables (402, 404, 406, 408) into a desired pose relative to the shaft (600A), a plurality of sensors (902, 904, 906, 908) for sensing tension exerted on the plurality of articulation cables (402, 404, 406, 408); and a controller (1050) in communication with the plurality of motors (802, 804, 806, 808) and the plurality of sensors (902, 904, 906, 908), the method comprising: (a) selecting the desired pose of the end effector (200) with respect to the shaft (600A); (b) conducting a surgical procedure on a tissue of a patient via the end effector (200) that causes tensions to be exerted on the plurality of articulation cables (402, 404, 406, 408) for the desired articulation pose of the end effector (200); (c) obtaining, by the controller (1050) via the plurality of sensors (902, 904, 906, 908), the tensions that are exerted on the plurality of articulation cables (402, 404, 406, 408) during the surgical procedure; (d) determining, by the controller (1050), whether any of the tensions exceed a first predetermined tension threshold; (e) in response to determining that any of the tensions exceed the first predetermined tension threshold, operating at least one motor of the plurality of motors (802, 804, 806, 808), by the controller (1050), to reduce the tension of at least one of the articulation cables (402, 404, 406, 408) until the tensions are at or below the first predetermined tension threshold; and (f) controlling, by the controller (1050), the operation of the at least one motor (802, 804, 806, 808) during reduction of the tension to substantially maintain the desired pose of the end effector (200).

### Example 2

The method of Example 1, wherein selecting the desired pose of the end effector (200) comprises operating at least one motor (802, 804, 806, 808) to articulate the end effector (200) with respect to the shaft (600A) into the desired pose.

### Example 3

The method of any one or more of Examples 1 and 2, wherein the end effector (200) includes a first jaw (182) and a second jaw (184) that cooperate to selectively clamp the tissue therebetween, and a knife (206) that is configured for selective actuation along a firing stroke relative to the first and second jaws (182, 184) and wherein the surgical procedure comprises one or more of clamping the tissue between the first and second jaws (182, 184) and actuating the knife (206) along the firing stroke.

### Example 4

The method of any one or more of Examples 1-3, wherein: (a) operating at least one motor of the plurality of motors (802, 804, 806, 808) comprises operating each motor of the plurality of motors (802, 804, 806, 808); and (b) controlling the operation of the at least one motor (802, 804, 806, 808) during the reduction of tension comprises indexing each motor of the plurality of motors (802, 804, 806, 808) in unison and by an amount determined to maintain the desired pose.

### Example 5

The method of any one or more of Examples 1-3, wherein: (a) obtaining the tensions exerted on the plurality of articulation cables (402, 404, 406, 408) comprises obtaining a respective tension exerted on each articulation cable of the plurality of articulation cables (402, 404, 406, 408); (b) operating at least one motor of the plurality of motors (802, 804, 806, 808) comprises individually indexing each motor of the plurality of motor (802, 804, 806, 808) until the respective tension of each articulation cable is below the first predetermined tension threshold; and (c) controlling operation of at least one motor (802, 804, 806, 808) comprises coordinating the indexing of each motor of the plurality of motors (802, 804, 806, 808) to substantially maintain the desired pose of the end effector (200).

### Example 6

The method of any one or more of Examples 1-5, further comprising, in response to determining that any of the tensions exceed the first predetermined tension threshold, calculating an offset position for the at least one motor (802, 804, 806, 808) that correlates to reducing the tension to at or below the first predetermined tension threshold, and wherein operating the at least one motor comprises indexing the at least one motor (802, 804, 806, 808) to the offset position.

### Example 7

The method of Example 1, further comprising (a) determining whether any of the tensions of the plurality of articulation cables (402, 404, 406, 408) fall below a second predetermined tension threshold, the second predetermined tension threshold being less than or equal to the first predetermined tension threshold; and (b) in response to determining that any of the tensions of the plurality of articulation cables (402, 404, 406, 408) fall below the second predetermined tension threshold, operating at least one motor of the plurality of motors (802, 804, 806, 808) to increase the tension of at least one of the articulation cables (402, 404, 406, 408) until the tensions are at or above the second predetermined tension threshold.

### Example 8

The method of Example 7, wherein: (a) operating at least one motor of the plurality of motors (802, 804, 806, 808) comprises operating each motor of the plurality of motors (802, 804, 806, 808); and (b) controlling the operation of the at least one motor (802, 804, 806, 808) during the increase of tension comprises indexing each motor of the plurality of motors (802, 804, 806, 808) in unison and by an amount determined to maintain the desired pose.

### Example 9

The method of any one or more of Examples 7-8, wherein (a) obtaining the tensions exerted on the plurality of articulation cables (402, 404, 406, 408) comprises obtaining a respective tension exerted on each articulation cable of the plurality of articulation cables (402, 404, 406, 408); (b) operating at least one motor of the plurality of motors (802, 804, 806, 808) comprises individually indexing each motor of the plurality of motors (802, 804, 806, 808) until the respective tension of each articulation cable is at or below the first predetermined tension threshold and is at or above the second predetermined tension threshold; and (c) controlling operation of at least one motor (802, 804, 806, 808) comprises coordinating the indexing of each motor of the plurality of motors (802, 804, 806, 808) to substantially maintain the desired pose of the end effector (200).

### Example 10

The method of any one or more of Examples 7-9, further comprising, in response to determining that the tensions exceed the first predetermined tension threshold or falls below the second predetermined position, calculating an offset position for the at least one motor (802, 804, 806, 808) that correlates to reducing the tensions to at or below the first predetermined tension threshold or increasing the tensions to at or above the second predetermined tension threshold, respectively, and wherein operating the at least one motor comprises indexing the at least one motors (802, 804, 806, 808) to the offset position.

### Example 11

The method of any one or more of Examples 7-10, wherein obtaining the tensions that are exerted on the plurality of articulation cables (402, 404, 406, 408) comprises sensing torque of the plurality of motors (802, 804, 806, 808) and extrapolating the tensions from the sensed torques.

### Example 12

A method for performing an electrosurgical procedure using a surgical system, wherein the surgical system includes an electrosurgical instrument (1000) having a shaft (600A), an articulation joint (300) coupled with the shaft (600A), an end effector (200) coupled with the articulation joint (300), a plurality of articulation cables (402, 404, 406, 408) coupled with the end effector (200) via the articulation joint (300) and manipulable to cause the end effector (200) to articulate at the articulation joint (300) with respect to the shaft (600A), a plurality of motors (802, 804, 806, 808) operably coupled with the plurality of articulation cables (402, 404, 406, 408) and configured to articulate the end effector (200) via the articulation cables (402, 404, 406, 408) into a desired pose relative to the shaft (600A), a plurality of sensors (902, 904, 906, 908) for sensing tension exerted on the plurality of articulation cables (402, 404, 406, 408); and a controller (1050) in communication with the plurality of motors (802, 804, 806, 808) and the plurality of sensors (902, 904, 906, 908), the method comprising (a) selecting the desired pose of the end effector (200) with respect to the shaft (600A); (b) predicting, by the controller (1050) via the plurality of sensors (902, 904, 906, 908), the tensions that are to be exerted on the plurality of articulation cables (402, 404, 406, 408) during the surgical procedure; (c) determining, by the controller (1050), the current position of the plurality of motors (802, 804, 806, 808) with the end effector (200) at the desired pose; (d) calculating an offset position for the plurality of motors (802, 804, 806, 808) based on the current position of the plurality of motors (802, 804, 806, 808) and the predicted tensions; (c) operating at least one motor of the plurality of motors (802, 804, 806, 808), by the controller (1050), to the offset position to reduce the tensions of the articulation cables (402, 404, 406, 408) to the predicted tensions; (f) controlling, by the controller (1050), the operation of the at least one motors (802, 804, 806, 808) during reduction of the tensions to substantially maintain the desired pose of the end effector (200); and (g) conducting a surgical procedure on a tissue of a patient via the end effector (200) at the predicted tensions.

### Example 13

The method of Example 12, wherein controlling the at least one motor of the plurality of motors (802, 804, 806, 808) during the reduction of tension comprises moving each of the motors (802, 804, 806, 808) of the plurality of motors (802, 804, 806, 808) in unison and by an amount determined to maintain the desired pose.

### Example 14

The method of any one or more of Examples 12-13, wherein predicting the tensions comprises predicting the tensions based on one or more of start time, end time, cable load, knife load, end effector pose, knife velocity, knife mechanism compliance, tissue thickness, or cable stiffness.

### Example 15

The method of any one or more of Examples 12-14, wherein selecting the desired pose of the end effector (200) comprises operating the at least one motor (802, 804, 806, 808) to articulate the end effector (200) with respect to the shaft (600A) into the desired pose.

The following clauses also relate to various non-exhaustive ways in which the teachings herein may be combined or applied.

Clause 1. A method for performing an electrosurgical procedure using an electrosurgical instrument, wherein the electrosurgical instrument includes a shaft, an articulation joint coupled with shaft, an end effector coupled with the shaft, a plurality of articulation cables coupled with the end effector via the articulation joint and manipulable to cause the end effector to articulate at the articulation joint with respect to the shaft, a plurality of motors operably coupled with the plurality of articulation cables and configured to articulate the end effector via the articulation cables into a desired pose relative to the shaft, a plurality of sensors for sensing tension exerted on the plurality of articulation cables; and a controller in communication with the plurality of motors and the plurality of sensors, the method comprising:
(a) selecting the desired pose of the end effector with respect to the shaft;
(b) conducting a surgical procedure on a tissue of a patient via the end effector that causes tensions to be exerted on the plurality of articulation cables for the desired articulation position of the end effector;
(c) obtaining, by the controller via the plurality of sensors, the tensions that are exerted on the plurality of articulation cables during the surgical procedure;
(d) determining, by the controller, whether any of the tensions exceed a first predetermined tension threshold;
(e) in response to determining that any of the tensions exceed the first predetermined tension threshold, operating at least one motor of the plurality of motors, by the controller, to reduce the tension of at least one of the articulation cables until the tensions are at or below the first predetermined tension threshold; and
(f) controlling, by the controller, the operation of the at least one motor during reduction of the tension to substantially maintain the desired pose of the end effector.

Clause 2. The method of Clause 1, wherein selecting the desired pose of the end effector comprises operating the at least one motor to articulate the end effector with respect to the shaft into the desired pose.

Clause 3. The method of Clause 1, wherein the end effector includes a first jaw and a second jaw that cooperate to selectively clamp the tissue therebetween, and a knife that is configured for selective actuation along a firing stroke relative to the first and second jaws and wherein the surgical procedure comprises one or more of clamping the tissue between the first and second jaws and actuating the knife along the firing stroke.

Clause 4. The method of Clause 1, wherein:
(a) operating at least one motor of the plurality of motors comprises operating each motor of the plurality of motors; and
(b) controlling the operation of the at least one motor during the reduction of tension comprises indexing each motor of the plurality of motors in unison and by an amount determined to maintain the desired pose.

Clause 5. The method of Clause 1, wherein:
(a) obtaining the tensions exerted on the plurality of articulation cables comprises obtaining a respective tension exerted on each articulation cable of the plurality of articulation cables;
(b) operating at least one motor of the plurality of motors comprises individually indexing each motor of the plurality of motor until the respective tension of each articulation cable is below the first predetermined tension threshold; and
(c) controlling operation of at least one motor comprises coordinating the indexing of each motor of the plurality of motors to substantially maintain the desired pose of the end effector.

Clause 6. The method of Clause 1, further comprising, in response to determining that any of the tensions exceed the first predetermined tension threshold, calculating an offset position for the at least one motor that correlates to reducing the tension to at or below the first predetermined tension threshold, and wherein operating the at least one motor comprises indexing the at least one motor to the offset position.

Clause 7. The method of Clause 1, further comprising:
(a) determining whether any of the tensions of the plurality of articulation cables fall below a second predetermined tension threshold, the second predetermined tension threshold being less than or equal to the first predetermined tension threshold; and
(b) in response to determining that any of the tensions of the plurality of articulation cables fall below the second predetermined tension threshold, operating at least one motor of the plurality of motors to increase the tension of at least one of the articulation cables until the tensions are at or above the second predetermined tension threshold.

Clause 8. The method of Clause 7, wherein:
(a) operating at least one motor of the plurality of motors comprises operating each motor of the plurality of motors; and
(b) controlling the operation of the at least one motor during the increase of tension comprises indexing each motor of the plurality of motors in unison and by an amount determined to maintain the desired pose.

Clause 9. The method of Clause 7, wherein:
(a) obtaining the tensions exerted on the plurality of articulation cables comprises obtaining a respective tension exerted on each articulation cable of the plurality of articulation cables;
(b) operating at least one motor of the plurality of motors comprises individually indexing each motor of the plurality of motors until the respective tension of each articulation cable is at or below the first predetermined tension threshold and is at or above the second predetermined tension threshold; and
(c) controlling operation of at least one motor comprises coordinating the indexing of each motor of the plurality of motors to substantially maintain the desired pose of the end effector.

Clause 10. The method of Clause 7, further comprising, in response to determining that the tensions exceed the first predetermined tension threshold or falls below the second predetermined position, calculating an offset position for the at least one motor that correlates to reducing the tensions to at or below the first predetermined tension threshold or increasing the tensions to at or above the second predetermined tension threshold, respectively, and wherein operating the at least one motor comprises indexing the at least one motors to the offset position.

Clause 11. The method of Clause 7, wherein obtaining the tensions that are exerted on the plurality of articulation cables comprises sensing torque of the plurality of motors and extrapolating the tensions from the sensed torques.

Clause 12. A method for performing an electrosurgical procedure using a surgical system, wherein the surgical system includes an electrosurgical instrument having a shaft, an articulation joint coupled with shaft, an end effector coupled with the shaft, a plurality of articulation cables coupled with the end effector via the articulation joint and manipulable to cause the end effector to articulate at the articulation joint with respect to the shaft, a plurality of motors operably coupled with the plurality of articulation cables and configured to articulate the end effector via the articulation cables into a desired pose relative to the shaft, a plurality of sensors for sensing tension exerted on the plurality of articulation cables; and a controller in communication with the plurality of motors and the plurality of sensors, the method comprising:
(a) selecting the desired pose of the end effector with respect to the shaft;
(b) predicting, by the controller via the plurality of sensors, the tensions that are to be exerted on the plurality of articulation cables during the surgical procedure;
(c) determining, by the controller, the current position of the plurality of motors with the end effector at the desired pose;
(d) calculating an offset position for the plurality of motors based on the current position of the plurality of motors and the predicted tensions;
(e) operating at least one motor of the plurality of motors, by the controller, to the offset position to reduce the tensions of the articulation cables to the predicted tensions;
(f) controlling, by the controller, the operation of the at least one motors during reduction of the tensions to substantially maintain the desired pose of the end effector; and
(g) conducting a surgical procedure on a tissue of a patient via the end effector at the predicted tensions.

Clause 13. The method of Clause 12, wherein controlling the at least one motor of the plurality of motors during the reduction of tension comprises moving each of the motors of the plurality of motors in unison and by an amount determined to maintain the desired pose.

Clause 14. The method of Clause 12, wherein predicting the tensions comprises predicting the tensions based on one or more of start time, end time, cable load, knife load, end effector pose, knife velocity, knife mechanism compliance, tissue thickness, or cable stiffness.

Clause 15. The method of Clause 12, wherein selecting the desired pose of the end effector comprises operating the at least one motor to articulate the end effector with respect to the shaft into the desired pose.

Clause 16. An apparatus comprising:
(a) an end effector operable to clamp, staple, and cut tissue, comprising:
   (i) a first jaw configured to selectively receive a staple cartridge,
   (ii) a second jaw, at least one of the first jaw and the second jaw being movable relative to each other to facilitate clamping of tissue therebetween, and
   (iii) a knife configured to move relative to the first and second jaws;
(b) an articulation joint coupled with the end effector and configured to articulate the end effector;
(c) a plurality of articulation cables coupled with the end effector via the articulation joint, the plurality of articulation cables being manipulable to cause the end effector to articulate;
(d) a plurality of motors associated with the plurality of articulation cables and configured to control movement of the plurality of articulation cables;
(e) a plurality of sensors associated with the plurality of motors and configured to sense tensions exerted on the plurality of tension cables by the plurality of motors; and
(f) a controller in communication with the plurality of motors and the plurality of sensors, the controller configured to:
   (i) obtain, using the plurality of sensors, the tensions exerted on the plurality of articulation cables during a surgical procedure,
   (ii) determine whether any of the tensions exerted on the articulation cables exceeds a predetermined tension threshold,
   (iii) in response to determining that any of the tensions exceed the predetermined tension threshold, operating at least one motor of the plurality of motors to reduce the tension of at least one of the articulation cables until the tensions are at or below the predetermined tension threshold, and
   (iv) controlling the operation of the at least one motor during reduction of the tension to substantially maintain the desired pose of the end effector.

Clause 17. The apparatus of Clause 16, wherein:
(a) operating at least one motor of the plurality of motors comprises operating each motor of the plurality of motors; and
(b) controlling the operation of the at least one motor during the reduction of tension comprises indexing each motor of the plurality of motors in unison and by an amount determined to maintain the desired pose.

Clause 18. The apparatus of Clause 16, wherein:
(a) obtaining the tensions exerted on the plurality of articulation cables comprises obtaining a respective tension exerted on each articulation cable of the plurality of articulation cables;
(b) operating at least one motor of the plurality of motors comprises individually indexing each motor of the plurality of motor until the respective tension of each articulation cable is below the first predetermined tension threshold; and
(c) controlling operation of at least one motor comprises coordinating the indexing of each motor of the plurality of motors to substantially maintain the desired pose of the end effector.

Clause 19. The apparatus of Clause 16, wherein the controller is further configured to:
(a) determine whether any of the tensions of the plurality of articulation cables fall below a second predetermined tension threshold, the second predetermined tension threshold being less than or equal to the first predetermined tension threshold; and
(b) in response to determining that any of the tensions of the plurality of articulation cables fall below the second predetermined tension threshold, operate at least one motor of the plurality of motors to increase the tension of at least one of the articulation cables until the tensions are at or above the second predetermined tension threshold.

Clause 20. The apparatus of Clause 19, wherein:
(a) operating at least one motor of the plurality of motors comprises operating each motor of the plurality of motors; and
(b) controlling the operation of the at least one motor during the increase of tension comprises indexing each motor of the plurality of motors in unison and by an amount determined to maintain the desired pose.

### IV. Miscellaneous

It should be understood that any one or more of the teachings, expressions, versions, examples, etc. described herein may be combined with any one or more of the other teachings, expressions, versions, examples, etc. that are described herein. The above-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as those made available by Auris Health, Inc. of Redwood City, CA or by Intuitive Surgical, Inc., of Sunnyvale, California.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1-15. (canceled)

16. A method for operating a surgical instrument that includes a shaft, an articulation joint operably coupled with shaft, an end effector operably coupled with the articulation joint, a plurality of articulation cables operably coupled with the end effector via the articulation joint, a plurality of motors operably coupled with the plurality of articulation cables, a plurality of sensors, and a controller in communication with the plurality of motors and the plurality of sensors, the method comprising:
positioning the end effector in a desired pose with respect to the shaft;
conducting a procedure with the end effector that causes tensions to be exerted on the plurality of articulation cables;
obtaining, by the controller via the plurality of sensors, the tensions that are exerted on the plurality of articulation cables during the procedure;
determining, by the controller, whether any of the tensions exceed a first predetermined tension threshold;
in response to determining that any of the tensions exceed the first predetermined tension threshold, operating at least one motor of the plurality of motors, by the controller, to reduce at least one tension of the tensions until the at least one tension is at or below the first predetermined tension threshold; and
controlling, by the controller, the at least one motor to substantially maintain the desired pose of the end effector while reducing the at least one tension.

17. The method of claim 16, wherein selecting the desired pose of the end effector comprises operating the at least one motor to articulate the end effector with respect to the shaft into the desired pose.

18. The method of claim 16, wherein the end effector includes a first jaw and a second jaw that cooperate to selectively clamp the tissue therebetween, and a knife that is configured for selective actuation along a firing stroke relative to the first and second jaws and wherein the surgical procedure comprises one or more of clamping the tissue between the first and second jaws and actuating the knife along the firing stroke.

19. The method of claim 16, wherein:
operating at least one motor of the plurality of motors comprises operating each motor of the plurality of motors; and
controlling the operation of the at least one motor during the reduction of tension comprises indexing each motor of the plurality of motors in unison and by an amount determined to maintain the desired pose.

20. The method of claim 16, wherein:
obtaining the tensions exerted on the plurality of articulation cables comprises obtaining a respective tension exerted on each articulation cable of the plurality of articulation cables;
operating at least one motor of the plurality of motors comprises individually indexing each motor of the plurality of motor until the respective tension of each articulation cable is below the first predetermined tension threshold; and
controlling operation of at least one motor comprises coordinating the indexing of each motor of the plurality of motors to substantially maintain the desired pose of the end effector.

21. The method of claim 16, further comprising, in response to determining that any of the tensions exceed the first predetermined tension threshold, calculating an offset position for the at least one motor that correlates to reducing the tension to at or below the first predetermined tension threshold, and wherein operating the at least one motor comprises indexing the at least one motor to the offset position.

22. The method of claim 16, further comprising:
determining whether any of the tensions of the plurality of articulation cables fall below a second predetermined tension threshold, the second predetermined tension threshold being less than or equal to the first predetermined tension threshold; and
in response to determining that any of the tensions of the plurality of articulation cables fall below the second predetermined tension threshold, operating at least one motor of the plurality of motors to increase the tension of at least one of the articulation cables until the tensions are at or above the second predetermined tension threshold.

23. The method of claim 22, wherein:
operating at least one motor of the plurality of motors comprises operating each motor of the plurality of motors; and
controlling the operation of the at least one motor during the increase of tension comprises indexing each motor of the plurality of motors in unison and by an amount determined to maintain the desired pose.

24. The method of claim 22, wherein:
obtaining the tensions exerted on the plurality of articulation cables comprises obtaining a respective tension exerted on each articulation cable of the plurality of articulation cables;
operating at least one motor of the plurality of motors comprises individually indexing each motor of the plurality of motors until the respective tension of each articulation cable is at or below the first predetermined tension threshold and is at or above the second predetermined tension threshold; and
controlling operation of at least one motor comprises coordinating the indexing of each motor of the plurality of motors to substantially maintain the desired pose of the end effector.

25. The method of claim 22, further comprising, in response to determining that the tensions exceed the first predetermined tension threshold or falls below the second predetermined position, calculating an offset position for the at least one motor that correlates to reducing the tensions to at or below the first predetermined tension threshold or increasing the tensions to at or above the second predetermined tension threshold, respectively, and wherein operating the at least one motor comprises indexing the at least one motors to the offset position.

26. The method of claim 22, wherein obtaining the tensions that are exerted on the plurality of articulation cables comprises sensing torque of the plurality of motors and extrapolating the tensions from the sensed torques.

27-35. (canceled)
